(19) United States
(12) Patent Application Publication
(10) Pub. No.: US 2023/0348920 A1
(43) Pub. Date: Nov. 2, 2023

(54) BOOSTING HOMOLOGY DIRECTED REPAIR IN PLANTS

(71) Applicant: KWS SAAT SE & Co. KGaA, Einbeck (DE)

(72) Inventor: Yu MEI, Olivette, MO (US)

(73) Assignee: KWS SAAT SE & Co. KGaA, Einbeck (DE)

(21) Appl. No.: 18/013,139

(22) PCT Filed: Jun. 29, 2021
(86) PCT No.: PCT/EP2021/067930

§ 371 Date: Dec. 27, 2022

Related U.S. Application Data
(60) Provisional application No. 63/045,487, filed on Jun. 29, 2020.

(51) Int. Cl. C12N 15/82; C12N 9/22; C12N 15/11

(52) U.S. Cl. CPC C12N 15/8213; C12N 9/22; C12N 15/111; C12N 15/11; C12N 2310/20; C12N 2800/80

(57) ABSTRACT

The present invention relates to the technical field of targeted modification of a nucleotide sequence of interest in the genome of a plant by specifically boosting homology-directed repair (HDR)-mediated genome editing. Provided are methods using at least one plant-specific HDR booster, at least one genome modification system and at least one repair template, optionally in combination with at least one plant regeneration booster, wherein such modified plant cells are regenerated in a direct or an indirect way. Finally, methods, tools, constructs and strategies are provided to effectively modify at least one genomic target site in a plant cell in a highly controllable manner to obtain said modified cell and to regenerate a plant tissue, organ, plant or seed from such modified cell.

### TECHNICAL FIELD

The present invention relates to the technical field of targeted modification of a nucleotide sequence of interest in the genome of a plant by specifically boosting homology-directed repair (HDR)-mediated genome editing. Provided are methods using at least one plant-specific HDR booster, at least one genome modification system and at least one repair template, optionally in combination with at least one plant regeneration booster, wherein such modified plant cells are regenerated in a direct or an indirect way. Finally, methods, tools, constructs and strategies are provided to effectively modify at least one genomic target site in a plant cell in a highly controllable manner to obtain said modified cell and to regenerate a plant tissue, organ, plant or seed from such modified cell.

### BACKGROUND OF THE INVENTION

While for many crops the use of CRISPR-based genome editing is being tested more and more frequently, little is known about successful and efficient methods for complex modifications via HDR-mediated genome editing. In other words, the use of the CRISPR machinery as such for certain types of plants and plant cells is meanwhile used quite often, but there is still the huge problem that there is a lack of knowledge of how to control a precise repair after a targeted genomic break has been made in a highly controllable manner.

Most genome editing (GE) techniques rely on the introduction of targeted double strand breaks (DSBs) or single strand breaks (SSBs) by means of a site-specific nuclease or nickase, for example of a known CRISPR system. The DSBs stimulate different cellular DNA repair pathways such as non-homologous end joining (NHEJ) or homology directed repair (HDR, also often referred to as homologous recombination (HR)) resulting in targeted modifications. NHEJ mostly causes random base pair insertions or deletions (InDels) that can lead to gene knockouts by disruption. In rarer cases, NHEJ repair mechanisms can also lead to the targeted introduction of a donor template, also called repair template (RT) having fitting ends (blunt or sticky ends) that allow integration of the donor DNA as encoded/provided on the RT. Therefore, NHEJ does not provide for an indeed controllable strategy to provide not only a targeted DSB/SSB, but also a targeted repair resulting in an inheritable genomic modification of actual interest.

HDR-based repair of the DSB/SSB can occur if a RT is provided that has matching regions to the target sequence (homologous arms). The homology region can also be microhomologies leading to microhomology-mediated end joining (MMEJ)) framing the sequence to be edited or inserted. Such HDR-based methods can create very precise insertions or base pair substitutions at the target site. This includes targeted insertion of larger nucleotide molecules, e.g. entire genes, or regulatory elements or the replacement of one allele for another, which for example carries exchange of one or more individual amino acid substitutions.

However, in most organisms—including plants— HDR-mediated GE is technically the more challenging way of achieving targeted editing because of the strong cellular prevalence of the NHEJ repair pathway (Puchta, Holger, J. Exp. Bot., 2005, 56(409):1-14). In most cell cycle phases NHEJ is the more common DNA repair mechanism leading to overall very low frequencies of targeted integration by HDR.

Several approaches and studies were conducted focusing on enhancing the HDR repair pathway in cells in the context of targeted GE, in particular GE using a CRISPR system for various eukaryotic systems.

For example, Charpentier et al. (Nat. Commun. 2018, 9(1):1133, doi: 10.1038/s41467-018-03475-7) report that fusing CtIP to the nuclease Cas9 enhances HDR-mediated transgene integration in human cells. To enable the generation of a functional fusion construct the functional fragment of CtIP has been identified. They demonstrated that only the N-terminal fragment of CtIP (amino acids (aa) 1-296) is needed to confer this effect. A rather long double stranded repair template had to be used together with a specific fusion protein. Also in the human system, Richardson et al. (Nature Genetics, 2018, vol. 50, 1132-1139) report that the inhibition of CtIP in human cells decreases HDR-mediated genome editing, but specifically in the context of the Fanconi anemia pathway in human cells.

All these data, however, indicate that special solutions have to be identified when intending to optimize HDR-mediated GE in plant cells in view of the fact that DNA repair pathways in plants are rather complex and different to mammalian/human DNA repair systems.

Wang et al. (Front. Plant Sci., 2018, 9, 1005) identified ZmCom1 being an orthologue of CtIP and observed defects in both mitotic and meiotic recombination, when the ZmCom1 protein is knocked-out. The authors thus described the natural function of ZmCom1 and its role in mitotic and meiotic processes, but they did not study or realize the suitability of this protein for GE purposes.

Overall, the sequence identity between the human CtIP and the corn ZmCom1 lies below 10% and, in particular, the corn ZmCom1 sequence does not possess a counterpart to the N-terminal fragment of CtIP that has been shown to trigger the boosting effect as reported in Charpentier et al., 2018 in mammalian cells. Therefore, identifying HDR-assisting proteins suitable for GE based on data acquired in mammalian/human systems is rather difficult, as in silico prediction will not provide straightforward results. No increased HDR-mediated gene editing efficiencies using CtIP/Com1 have been demonstrated for plants. Therefore, the disclosed approaches per se are not suitable for using them to efficiently increase HDR-mediated GE efficiencies in plants.

As Arabidopsis thaliana is a well-known and suitable target organism, several studies were conducted to elucidate the natural repair processes in this model plant. Shaked et al. (Proc. Natl. Acad. Sci., 2005 Aug. 23; 102(34): 12265-12269.) found that stable expression of the RAD54 gene from yeast enhances the gene-targeting frequency in transgenic Arabidopsis lines. Still, no plant endogenous genes or the concerted action thereof during RT-based GE was studied. In a further study in Arabidopsis thaliana, Seeliger et al. (New Phytol., 2012, 193(2):364-75) demonstrate that AtBRCA2 is important for both somatic and meiotic homologous recombination based on phenotype analysis of the AtBRCA2 double mutant. However, gene editing using this protein for enhancing HDR was not demonstrated or suggested.

Finally, Zhang et al. (J. Exp. Bot., 2015, 66 (19): 5713) studied the role of XRCC3 for DSB repair and HDR in rice and it has been demonstrated XRCC3 is essential for proper double-strand break repair and homologous recombination during rice meiosis. A xrcc3 knock-out mutant showed defects in DSB repair and homologous chromosome recombination during meiosis. Zhang et al. did not transfer these findings to GE settings, or to other relevant crop plants.

In all of the above studies, it was found that the HDR enhancing proteins cannot easily be interchanged between species in view of the conserved and crucial mechanisms naturally occurring during DNA damage repair as such representing a relevant process for living cells. Although many efforts have been made to increase targeted and precise HDR-mediated gene editing in mammalian cells and in certain model organisms, at date little is known about increasing HDR-mediated gene editing efficiencies in many important crops such as corn, for example.

Presently, there is thus a great need in providing simple methods that allow boosting the frequency of efficient and precise HDR-mediated modifications of nucleic acids of interest in particular for the use in crop plants. Further, there is a great need in identifying suitable HDR-assisting or -boosting proteins having their origin in plant cells, and as such being optimized for DNA repair mechanisms in plant cells. Further, there is a great need in optimizing GE in plant cells, particularly in relevant crop plants, to achieve more reliable and site-specific GE.

It was thus an object of the present invention to provide new generally applicable HDR boosting tools for gene editing in plants and to define new strategies for the use of these HDR boosting effectors, preferably being of plant origin, during RT-based gene editing for precisely modifying target sites of interest in a plant cell's genome by controlling not only the DSB/SSB, but additionally the subsequent repair.

### SUMMARY OF THE INVENTION

To address the above objectives, the present invention thus provides, in a first aspect, a method for the targeted modification of at least one genomic target sequence in at least one plant cell, wherein the method comprises the following steps: (a) providing at least one plant cell to be modified; (b) introducing into the cell: (i) at least one plant-specific HDR booster, or a sequence encoding the same, or an orthologue, paralogue, homologue, or an active fragment thereof, or a sequence encoding the same, or a combination of at least two plant-specific HDR boosters, preferably wherein the at least one plant HDR booster comprises a consensus motif according to SEQ ID NOs: 91 to 95; (ii) at least one genome editing system comprising at least one site-specific nuclease or site-specific nickase, or a sequence encoding the same, and optionally, in the case a CRISPR system is used, at least one guide molecule, or a sequence encoding the same; and (iii) at least one repair template, or a sequence encoding the same; (c) cultivating the at least one cell under conditions allowing the expression and/or assembly of the at least one plant HDR booster, the at least one genome editing system, and the at least one repair template; and (d) obtaining at least one modified cell; and (e) optionally: obtaining at least one plant, plant tissue, organ, or seed regenerated from the at least one modified cell.

In certain embodiments, the method further comprises an additional step following either step (d) or (e) comprising: (f) screening for at least one modified plant, plant cell, plant tissue, organ, or seed carrying a desired targeted modification.

In other embodiments, the method further comprises during step (b) (iv) providing at least one regeneration booster, or a sequence encoding the same, for promoting plant cell proliferation to assist a targeted modification of at least one genomic target sequence, optionally after expression of the regeneration booster.

In certain embodiments of the above methods, the at least one plant-specific HDR booster, or the orthologue, paralogue, homologue, or active fragment thereof, or the nucleic acid sequence encoding the same, is independently selected from a plant-specific COM1, ExoI, XRCC3, Radx, BRCA2, ZmChr18, or a RecQ helicase protein, or any combination thereof.

In certain embodiments of the above methods, the at least one plant-specific HDR booster is independently selected from the group consisting of SEQ ID NOs: 24 to 30, 33, 35, 37, 39, 41, 43, 45, 47, 49, 51, 53, 55, 57, 59, 61, 63, 65, 67, 69, 71, 73, 75, 78 to 90, or 120, or a sequence having at least 90%, 91%, 92%, 93%, 94%, 95%, 96%, 97%, 98% or 99% sequence identity thereto, or an orthologue, paralogue, homologue, or an active fragment thereof, or a nucleic acid sequence encoding the same.

According to certain embodiments of the above methods, the nucleic acid sequence encoding the at least one plant-specific HDR booster is selected from the group consisting of SEQ ID NOs: 5 to 11, 32, 34, 36, 38, 40, 42, 44, 46, 48, 50, 52, 54, 56, 58, 60, 62, 64, 66, 68, 70, 72, 74, or 119, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, provided that the sequence encodes a corresponding plant-specific HDR booster with an enzymatic HDR booster activity as defined above.

In one embodiment of the above methods, the at least one genome editing system, the at least one repair template and/or the at least one regeneration booster, or the sequence(s) encoding the same, is/are provided prior to, simultaneously with, or subsequently to providing the at least one plant-specific HDR booster.

In another embodiment of the above methods, the method comprises an intermediate regeneration step before obtaining at least one modified cell, and the regeneration step comprises direct meristem organogenesis, or the regeneration step comprises a step of indirect callus embryogenesis or organogenesis.

In yet another embodiment of the above methods, the at least one plant-specific HDR booster, the at least one genome editing system, the at least one regeneration booster and/or the at least one repair template, or the sequences encoding the same, are introduced into the cell by transformation or transfection mediated by biolistic bombardment, Agrobacterium-mediated transformation, micro- or nanoparticle delivery, chemical transfection, or a combination thereof, preferably wherein the introduction is mediated by biolistic bombardment, preferably wherein the biolistic bombardment comprises a step of osmotic treatment before and/or after bombardment.

In certain embodiments of the above methods, the at least one genome editing system is selected from a CRISPR/Cas system, preferably from a CRISPR/MAD7 system, a CRISPR/Cpf1 (CRISPR/Cas12a) system, a CRISPR/MAD2 system, a CRISPR/Cas9 system, a CRISPR/CasX system, a CRISPR/CasY system, a CRISPR/Cas13 system, or a CRISPR/Csm system, or wherein the at least one site-directed nuclease or nickase, or a sequence encoding the same, is selected from a zinc finger nuclease system, or a transcription activator-like nuclease system, or a meganuclease system, or any combination, variant, or an active fragment thereof.

In one embodiment, the at least one genome editing system further comprises at least one reverse transcriptase and/or at least one cytidine or adenine deaminase, preferably wherein the at least one cytidine or adenine deaminase is independently selected from an apolipoprotein B mRNA-editing complex (APOBEC) family deaminase, preferably a rat-derived APOBEC, an activation-induced cytidine deaminase (AID), an ACF1/ASE deaminase, an ADAT family deaminase, an ADAR2 deaminase, a PmCDA1 deaminase, a TadA derived deaminase, and/or a transposon, or a sequence encoding the aforementioned at least one enzyme, or any combination, variant, or an active fragment thereof.

In certain embodiments of the above methods, the at least one repair template comprises or encodes a double- and/or single-stranded nucleic acid sequence.

In one embodiment of the above methods, the at least one repair template comprises symmetric or asymmetric homology arms, and/or the at least one repair template comprises at least one chemically modified base and/or backbone. The length of the at least one homology arm, independently for the 5′ and/or a 3′ homology arm relative to the insert repair template sequence in between, may vary from no homology arm at all, a short length homology arm from around 1 or two base pair(s) (bp) to around 70 bp, for a medium length homology arm from around 70 base pairs (bp) to around 500 bp, e.g., and for a long length homology arm from around 500 bp to up to several kbp, preferably from around 50 bp to around 1 kb. This applies for any kind of repair template, in case this repair template is designed to comprise homology arms.

In embodiments, where asymmetric homology arms are used, the length of the longer and the shorter homology arm and the 5′/3′ positioning thereof within the repair template in its overall length will, for example, depend on the nucleic acid guided nuclease (or variant thereof) of interest and its binding and release mode of cut (genomic) DNA so that the asymmetry of the homology arms is particularly in a way that asymmetry allows easy access of the repair template to the cut target site by early entering and annealing at that portion released first by the nucleic acid guided nuclease after inserting a single or double-stranded cut or break.

In another embodiment of the above methods, at least one regeneration booster is provided and the regeneration booster comprises at least one of an RBP encoding sequence and/or at least one PLT encoding sequence, preferably wherein the regeneration booster comprises at least one of an RBP encoding sequence, wherein the at least one regeneration booster sequence is individually selected from any one of SEQ ID NOs: 96 to 106 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or an active fragment thereof, or the at least one regeneration booster sequence is encoded by a sequence individually selected from any one of SEQ ID NOs: 4 and 107 to 116, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, provided that the sequence encodes the respective regeneration booster according to SEQ ID NOs: 96 to 106 or an active fragment thereof, and optionally at least one further regeneration booster is introduced, wherein the further regeneration booster, or the sequence encoding the same is selected from BBM, WUS, WOX, RKD4, RKD2, GRF, LEC, or a variant or active fragment thereof.

In yet another embodiment of the above methods, the regeneration booster comprises at least one first RBP or PLT sequence, or a sequence encoding the same, preferably at least one RBP sequence, or the sequence encoding the same, and the regeneration booster further comprises: (i) at least one further RBP and/or PLT sequence, or the sequence encoding the same, or a variant thereof, (ii) at least one BBM sequence, or the sequence encoding the same, or a variant thereof, (iii) at least one WOX sequence, including WUS1, WUS2, or WOX5, or the sequence encoding the same, or a variant thereof, (iv) at least one RKD4 or RKD2 sequence, including wheat RKD4, or the sequence encoding the same, or a variant thereof, (v) at least one GRF sequence, including Zea mays GRF5 and Zea mays TOW/GRF1, or the sequence encoding the same, or a variant thereof, and/or (vi) at least one LEC sequence, including LEC1 and LEC2, or the sequence encoding the same, or a variant thereof, and wherein the at least one second regeneration booster, or a sequence encoding the same, is different to the first regeneration booster.

In another embodiment of the above methods, the at least one plant-specific HDR booster, the at least one genome editing system, the at least one repair template, and optionally the at least one regeneration booster, or the respective sequences encoding the same, are introduced transiently or stably, or as a combination thereof.

In a further aspect, there is provided a plant, plant cell, tissue, organ, or seed obtainable by or obtained by a method according to any of the preceding claims.

In certain embodiments, the plant, plant cell, tissue, organ, or seed is of a monocotyledonous or of a dicotyledonous plant.

In certain embodiments, the plant is selected from a plant originating from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant or plant cell originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia foumieri, Allium cepa, Allium fistulosum, Allium sativum, Allium tuberosum, Helianthus annuus, Helianthus tuberosus and/or Spinacia oleracea.

In yet a further aspect, there is provided an expression construct assembly, comprising: (i) at least one vector encoding at least one plant-specific HDR booster, preferably wherein the plant-specific HDR booster is as defined according to the embodiments of the first aspect above, (ii) at least one vector encoding at least one genome editing system, preferably wherein the genome editing system is as defined according to the embodiments of the first aspect above, optionally comprising at least one vector encoding at least one guide molecule as defined according to the first aspect above guiding the at least one nucleic acid guided nuclease or nickase to the at least one genomic target site of interest; (iii) optionally: at least one vector encoding at least one repair template, preferably wherein the repair template is as defined according to the embodiments of the first aspect above; and (iv) optionally: at least one vector encoding at least one regeneration booster, preferably wherein the regeneration booster is as defined according to the embodiments of the first aspect above; wherein (i), (ii), (iii), and/or (iv) are encoded on the same, or on different vectors.

The present invention will now be further described in detail based on the Figures, Sequences and the Detailed Description and Examples provided below.

### Short Description of Sequences

The sequences according to the sequence listing are briefly characterized in detail below:

| NO | Description |
|---|---|
| 1 | Single stranded oligo repair template for m7GEP22 target (rtGEP54) |
| 2 | Coding sequence of MAD7 endonuclease including NLSs at the N- and |
| | C- terminus |
| 3 | crRNA for m7GEP22 target in HMG13 gene, expressed in the construct |
| | flanked by ribozyme HH and HDV |
| 4 | Coding sequence of regeneration booster protein 2 (RBP2) codon |
| | optimized for Zea mays |
| 5 | Coding sequence of ZmRad52, N-terminus deleted to remove the |
| | predicted mitochondria localization signal |
| 6 | Coding sequence ZmBRCA2 |
| 7 | Coding sequence of ZmCOM1 |
| 8 | Coding sequence of ZmRad54 |
| 9 | Coding sequence of ZmXRCC3 |
| 10 | Coding sequence of ZmExol |
| 11 | Coding sequence of RecQ helicase RecQI4 codon optimized for Zea |
| | mays |
| 12 | Sequence of the vector GEMT130, including expression cassette ZmUbi- |
| | intron:NosT |
| 13 | Sequence of the promotor ZmUbi plus intron |
| 14 | Sequence of the vector pGEP1054, including expression cassette BdUbi- |
| | intron:MAD7:NosT |
| 15 | Sequence of the vector pGEP1067, including expression cassette |
| | ZmUbi-intro:crRNAm7GEP22:NosT |
| 16 | Sequence of the vector pGEP949, including expression cassette BdEF1- |
| | intron:KWS-RBP2:NosT |
| 17 | Sequence of the vector GEMT158, including expression cassette ZmUbi- |
| | intron:Rad52a:NosT |
| 18 | Sequence of the vector GEMT159, including expression cassette ZmUbi- |
| | intron:BRCA2:NosT |
| 19 | Sequence of the vector GEMT160, including expression cassette ZmUbi- |
| | intron:Com1:NosT |
| 20 | Sequence of the vector GEMT161, including expression cassette ZmUbi- |
| | intron:Rad54:NosT |
| 21 | Sequence of the vector GEMT162, including expression cassette ZmUbi- |
| | intron:XRCC3:NosT |
| 22 | Sequence of the vector GEMT188, including expression cassette ZmUbi- |
| | intronRecQI4:NosT |
| 23 | Sequence of the vector GEMT215, including expression cassette ZmUbi- |
| | intron:Exol:NosT |
| 24 | Protein sequence of ZmRad52, N-terminus deleted to remove the |
| | predicted mitochondria localization signal |
| 25 | Protein sequence ZmBRCA2 |
| 26 | Protein sequence of ZmCOM1 |
| 27 | Protein sequence of ZmRad54 |
| 28 | Protein sequence of ZmXRCC3 |
| 29 | Protein sequence of ZmExol |
| 30 | Protein sequence of RecQI4 |
| 31 | HiBiT tag 33 nt + stop codon = 36 nt |
| 32 | Sugar beet XRCC3 coding sequence |
| 33 | Sugar beet XRCC3 protein sequence |
| 34 | Wheat XRCC3 coding sequence |
| 35 | Wheat XRCC3 protein sequence |
| 36 | Rapeseed XRCC3 coding sequence |
| 37 | Rapeseed XRCC3 protein sequence |
| 38 | Potato XRCC3 coding sequence |
| 39 | Potato XRCC3 protein sequence |
| 40 | Sugar beet COM1 coding sequence |
| 41 | Sugar beet COM1 protein sequence |
| 42 | Wheat COM1 coding sequence |
| 43 | Wheat COM1 protein sequence |
| 44 | Rapeseed COM1 coding sequence |
| 45 | Rapeseed COM1 protein sequence |
| 46 | Potato COM1 coding sequence |
| 47 | Potato COM1 protein sequence |
| 48 | Sugar beet Exol coding sequence |
| 49 | Sugar beet Exol protein sequence |
| 50 | Wheat Exol coding sequence |
| 51 | Wheat Exol protein sequence |
| 52 | Rapeseed Exol coding sequence |
| 53 | Rapeseed Exol protein sequence |
| 54 | Potato Exol coding sequence |
| 55 | Potato Exol protein sequence |
| 56 | Sugar beet Rad54 coding sequence |
| 57 | Sugar beet Rad54 protein sequence |
| 58 | Wheat Rad54 coding sequence |
| 59 | Wheat Rad54 protein sequence |
| 60 | Rapeseed Rad54 coding sequence |
| 61 | Rapeseed Rad54 protein sequence |
| 62 | Sugar beet Rad52 coding sequence |
| 63 | Sugar beet Rad52 protein sequence |
| 64 | Wheat Rad52 coding sequence |
| 65 | Wheat Rad52 protein sequence |
| 66 | Rapeseed Rad52 coding sequence |
| 67 | Rapeseed Rad52 protein sequence |
| 68 | Potato Rad52 coding sequence |
| 69 | Potato Rad52 protein sequence |
| 70 | Sugar beet BRCA2 coding sequence |
| 71 | Sugar beet BRCA2 protein sequence |
| 72 | Wheat BRCA2 coding sequence |
| 73 | Wheat BRCA2 protein sequence |
| 74 | Rapeseed BRCA2 coding sequence |
| 75 | Rapeseed BRCA2 protein sequence |
| 76 | crRNA for crGEP43 target in Glyk gene, expressed in the construct |
| | flanked by ribozyme HH and HDV |
| 77 | Single stranded oligo repair template for crGEP43 target site in the Glyk |
| | gene (rtGEP56) |
| 78 | Soybean COM1 orthologue protein sequence |
| 79 | Rice COM1 orthologue protein sequence |
| 80 | Grape COM1 orthologue protein sequence |
| 81 | Rice Exo1 orthologue protein sequence |
| 82 | Arabidopsis Exo1 orthologue protein sequence |
| 83 | Arabidopsis Rad54 orthologue protein sequence |
| 84 | Chlamydomonas Rad54 orthologue protein sequence |
| 85 | Soybean Rad54 orthologue protein sequence |
| 86 | Rice Rad54 orthologue protein sequence |
| 87 | Grape Rad54 orthologue protein sequence |
| 88 | Rice XRCC3 orthologue protein sequence |
| 89 | Grape XRCC3 orthologue protein sequence |
| 90 | Soybean XRCC3 orthologue protein sequence |
| 91 | Consensus sequence or motif COM1 |
| 92 | Consensus sequence or motif Exo1 |
| 93 | Consensus sequence or motif Rad54 |
| 94 | Consensus sequence or motif BRCA2, wherein Xaa at position 2 is |
| | preferably selected from P or H, and wherein Xaa at position 5 is |
| | preferably selected from T, A, S, N, or Q. |
| 95 | Consensus sequence or motif XRCC3 |
| 96 | Artificial sequence RBP1 (regeneration booster protein 1) |
| 97 | RBP2 encoded by SEQ ID NO: 4 |
| 98 | Artificial sequence RBP3 |
| 99 | Artificial sequence RBP4 |
| 100 | Artificial sequence RBP5 |
| 101 | Artificial sequence RBP6 |
| 102 | Artificial sequence RBP7 |
| 103 | Artificial sequence RBP8 |
| 104 | Protein Zea mays ZmPLT3 |
| 105 | Protein Zea mays ZmPLT5 |
| 106 | Protein Zea Mays ZmPLT7 |
| 107 | DNA RBG1 (regeneration booster gene 1 encoding regeneration booster |
| | protein RBP1 according to SEQ ID NO: 96) |
| 108 | DNA Artificial sequence RBG3 encoding RBP3 |
| 109 | DNA Artificial sequence RBG4 encoding RBP4 |
| 110 | DNA Artificial sequence RBG5 encoding RBP5 |
| 111 | DNA Artificial sequence RBG6 encoding RBP6 |
| 112 | DNA Artificial sequence RBG7 encoding RBP7 |
| 113 | DNA Artificial sequence RBG8 encoding RBP8 |
| 114 | DNA Zea mays ZmPLT3 |
| 115 | DNA Zea mays ZmPLT5 |
| 116 | DNA Zea Mays ZmPLT7 |
| 117 | cDNA ZmTOW/GRF1 Artificial sequence |
| 118 | Protein ZmTOW/GRF1 |
| 119 | DNA Zea mays Chromatin remodelling factor 19 (ZmChr18) |
| 120 | Protein Zea mays Chromatin remodelling factor 19 (ZmChr18) |
| 121 | Artificial sequence rtGEP67 |

### BRIEF DESCRIPTION OF FIGURES

FIG. 1 shows a schematic of the m7GEP22 target site in the HMG13 gene including the PAM region, the repair template (rtGEP54 as single strand oligo repair template) and the different editing outcomes when repaired through either non-homologous end joining (NHEJ) or homology directed repair (HDR).

FIG. 2 shows an exemplary target and non-target strand of a target region including the PAM (protospacer adjacent motif) sequence as relevant for recognition by a CRISPR system. Single stranded oligonucleotides of the non-target strand sequence were used as repair template.

FIG. 3 shows efficiencies of HDR-mediated gene editing in corn using immature embryo derived type-II callus samples with either 200 ng (left) or 500 ng (right) repair template (rtGEP54). HDR-efficiencies were calculated as the ratio of HDR-mediated events over all mutations.

FIG. 4 shows efficiencies of HDR-mediated gene editing in corn using immature embryo derived type-II callus samples with 200 ng repair template (rtGEP54). To boost HDR efficiencies, several different positive regulators of the HDR pathway (e. g. ZmCOM1) were transiently overexpressed. HDR-efficiencies were calculated as the ratio of HDR-mediated events over all mutations.

FIG. 5 shows efficiencies of HDR-mediated gene editing in corn using immature embryo derived type-II callus samples with 500 ng repair template (rtGEP54). To boost HDR efficiencies, several different positive regulators of the HDR pathway (e. g. ZmCOM1, ZmXRCC3, or ZmExoI) were transiently overexpressed and the amount of repair template was increased. HDR-efficiencies were calculated as the ratio of HDR-mediated events over all mutations.

FIG. 6 shows an alignment of different (Potato, SEQ ID NO: 47; Rapeseed, SEQ ID NO: 45; Sugar beet, SEQ ID NO: 41, Wheat, SEQ ID NO: 43, Corn, SEQ ID NO: 26) COM1 orthologues. Sequence conservation is given in a bar diagram below the alignment. The identified sequence pattern is boxed.

FIG. 7 shows an alignment of different (Corn, SEQ ID NO: 29; Wheat, SEQ ID NO: 51; Rapeseed, SEQ ID NO: 53, Potato, SEQ ID NO: 55, Sugar beet, SEQ ID NO: 49) Exo1 orthologues. Sequence conservation is given in a bar diagram below the alignment. The identified sequence pattern is boxed.

FIG. 8 shows an alignment of different (Corn, SEQ ID NO: 27; Wheat, SEQ ID NO: 59; Sugar beet, SEQ ID NO: 57, Rapeseed, SEQ ID NO: 61) RAD54 orthologues. Sequence conservation is given in a bar diagram below the alignment. The identified sequence pattern is boxed.

FIG. 9 shows an alignment of different (Wheat, SEQ ID NO: 73; Corn, SEQ ID NO: 25; Sugar beet, SEQ ID NO: 71, Rapeseed, SEQ ID NO: 75) BRCA2 orthologues. Sequence conservation is given in a bar diagram below the alignment. The identified sequence pattern is boxed.

FIG. 10 shows an alignment of different (Wheat, SEQ ID NO: 35; Corn, SEQ ID NO: 28; Potato, SEQ ID NO: 39, Rapeseed, SEQ ID NO: 37; Sugar beet, SEQ ID NO: 33) XRCC3 orthologues. Sequence conservation is given in a bar diagram below the alignment. The identified sequence pattern is boxed.

FIG. 11 shows survey Tables summarizing the results of BLAST searches for various orthologues for Com1, Exo1, XRCC3, Rad54, and BRCA2 sequences identified and tested herein showing that these sequences only have a very low mutual sequence identity.

### DEFINITIONS

Unless defined otherwise, technical and scientific terms used herein have the same meaning as commonly understood by one of ordinary skill in the art to which this invention belongs.

As used in the context of the present application, the term “about” means+/−10% of the recited value, preferably +/−5% of the recited value. For example, about 100 nucleotides (nt) shall be understood as a value between 90 and 110 nt, preferably between 95 and 105 nt.

An “active fragment” in the context of a protein or enzyme as used herein refers to a truncated, i.e., shorter version of the respective full-length protein or enzyme, wherein the active fragment still comprises all relevant amino acids and folds into the correct structure so that it exerts the same core function of the respective full-length protein or enzyme. Active fragments may be preferred in certain settings, as the resulting proteins or enzymes are smaller and sterically less demanding.

A “base editor” as used herein refers to a protein or a fragment thereof having the same catalytic activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor. Usually, base editors are thus used as molecular complex. Base editors, including, for example, CBEs (base editors mediating C to T conversion) and ABEs (adenine base editors mediating A to G conversion), are powerful tools to introduce direct and programmable mutations without the need for double-stranded cleavage (Komor et al., Nature, 2016, 533(7603), 420-424; Gaudelli et al., Nature, 2017, 551, 464-471). In general, base editors are composed of at least one DNA targeting module and a catalytic domain that deaminates cytidine or adenine. All four transitions of DNA (A→T to G→C and C→G to T→A) are possible as long as the base editors can be guided to the target site. Originally developed for working in mammalian cell systems, both BEs and ABEs have been optimized and applied in plant cell systems. Efficient base editing has been shown in multiple plant species (Zong et al., Nature Biotechnology, vol. 25, no. 5, 2017, 438-440; Yan et al., Molecular Plant, vol. 11, 4, 2018, 631-634; Hua et al., Molecular Plant, vol. 11, 4, 2018, 627-630). Base editors have been used to introduce specific, directed substitutions in genomic sequences with known or predicted phenotypic effects in plants and animals. But they have not been used for directed mutagenesis targeting multiple sites within a genetic locus or several loci to identify novel or optimized traits.

A “CRISPR nuclease”, as used herein, is a specific form of a site-directed nuclease and refers to any nucleic acid guided nuclease which has been identified in a naturally occurring CRISPR system, which has subsequently been isolated from its natural context, and which preferably has been modified or combined into a recombinant construct of interest to be suitable as tool for targeted genome engineering. Any CRISPR nuclease can be used and optionally reprogrammed or additionally mutated to be suitable for the various embodiments according to the present invention as long as the original wild-type CRISPR nuclease provides for DNA recognition, i.e., binding properties. CRISPR nucleases also comprise mutants or catalytically active fragments or fusions of a naturally occurring CRISPR effector sequences, or the respective sequences encoding the same. A CRISPR nuclease may in particular also refer to a CRISPR nickase or even a nuclease-dead variant of a CRISPR polypeptide having endonucleolytic function in its natural environment. A variety of different CRISPR nucleases/systems and variants thereof are meanwhile known to the skilled person and include, inter alia, CRISPR/Cas systems, including CRISPR/Cas9 systems (EP2771468), CRISPR/Cpf1 (CRISPR/Cas12a) systems (EP3009511B1), CRISPR/C2C2 systems, CRISPR/CasX systems, CRISPR/CasY systems, CRISPR/Cmr systems, CRISPR/MAD systems, including, for example, CRISPR/MAD7 systems (WO2018236548A1) and CRISPR/MAD2 systems, CRISPR/CasZ systems and/or any combination, variant, or catalytically active fragment thereof. A nuclease may be a DNAse and/or an RNAse, in particular taking into consideration that certain CRISPR effector nucleases have RNA cleavage activity alone, or in addition to the DNA cleavage activity.

A “CRISPR system” is thus to be understood as a combination of a CRISPR nuclease or CRISPR effector, or a nickase or a nuclease-dead variant of said nuclease, or a functional active fragment or variant thereof together with the cognate guide RNA (or pegRNA or crRNA) guiding the relevant CRISPR nuclease. A “guide RNA” or “guide molecule” may be composed of a single molecule (a sgRNA), or it may comprise two separate molecules. Some CRISPR systems only need a crRNA to be active, whereas other CRISPR systems require the presence of a crRNA and a tracrRNA. These relevant RNA portions have to be incorporated into a guide molecule accordingly to guarantee a functional RNA-guided CRISPR system.

As used herein, the terms “(regeneration) booster”, “booster gene”, “booster polypeptide”, “boost polypeptide”, “boost gene” and “boost factor”, refer to a protein/peptide(s), or a (poly)nucleic acid fragment encoding the protein/polypeptide, causing improved plant regeneration of transformed or gene edited plant cells, which may be particularly suitable for improving genome engineering, i.e., the regeneration of a modified plant cell after genome engineering. Such protein/polypeptide may increase the capability or ability of a plant cell, preferably derived from somatic tissue, embryonic tissue, callus tissue or protoplast, to regenerate in an entire plant, preferably a fertile plant. Thereby, they may regulate somatic embryo formation (somatic embryogenesis) and/or they may increase the proliferation rate of plant cells. The regeneration of transformed or gene edited plant cells may include the process of somatic embryogenesis, which is an artificial process in which a plant or embryo is derived from a single somatic cell or group of somatic cells. Somatic embryos are formed from plant cells that are not normally involved in the development of embryos, i.e. plant tissue like buds, leaves, shoots etc. Applications of this process may include: clonal propagation of genetically uniform plant material; elimination of viruses; provision of source tissue for genetic transformation; generation of whole plants from single cells, such as protoplasts; development of synthetic seed technology. Cells derived from competent source tissue may be cultured to form a callus. Further, the term “regeneration booster” may refer to any kind of chemical having a proliferative and/or regenerative effect when applied to a plant cell, tissue, organ, or whole plant in comparison to a no-treated control. The particular artificially created regeneration booster polypeptides according to the present invention may have the dual function of increasing plant regeneration as well as increasing desired genome modification and gene editing outcomes.

As used herein, a “flanking region”, is a region of the repair nucleic acid molecule having a nucleotide sequence which is homologous to the nucleotide sequence of the DNA region flanking (i.e. upstream or downstream) of the preselected site.

A “genome” as used herein is to be understood broadly and comprises any kind of genetic information (RNA/DNA) inside any compartment of a living cell. In the context of a “genome modification”, the term thus also includes artificially introduced genetic material, which may be transcribed and/or translated, inside a living cell, for example, an episomal plasmid or vector, or an artificial DNA integrated into a naturally occurring genome.

The term of “genome engineering” as used herein refers to all strategies and techniques for the genetic modification of any genetic information (DNA and RNA) or genome of a plant cell, comprising genome transformation, genome editing, but also including less site-specific techniques, including TILLING and the like. As such, “genome editing” or “gene editing” (GE) more specifically refers to a special technique of genome engineering, wherein a targeted, specific modification of any genetic information or genome of a plant cell. As such, the terms comprise gene editing of regions encoding a gene or protein, but also the editing of regions other than gene encoding regions of a genome. It further comprises the editing or engineering of the nuclear (if present) as well as other genetic information of a plant cell, i.e., of intronic sequences, non-coding RNAs, miRNAs, sequences of regulatory elements like promoter, terminator, transcription activator binding sites, cis or trans acting elements. Furthermore, “genome engineering” also comprises an epigenetic editing or engineering, i.e., the targeted modification of, e.g., DNA methylation or histone modification, such as histone acetylation, histone methylation, histone ubiquitination, histone phosphorylation, histone sumoylation, histone ribosylation or histone citrullination, possibly causing heritable changes in gene expression.

A “genome modification system” as used herein refers to any DNA, RNA and/or amino acid sequence introduced into the cell, on a suitable vector and/or coated on a particles and/or directly introduced. A “genome editing” system more specifically refers to any DNA, RNA and/or amino acid sequence introduced into the cell, on a suitable vector and/or coated on a particles and/or directly introduced, wherein the “genome editing system” comprises at least one component being, encoding, or assisting a site-directed nuclease, nickase or inactivated variant thereof in modifying and/or repairing a genomic target site.

A “genomic target sequence” as used herein refers to any part of the nuclear and/or organellar genome of a plant cell, whether encoding a gene/protein or not, which is the target of a site-directed genome editing or gene editing experiment.

The term “homologous” as used herein refers to a certain degree of correspondence, similarity, or identity of two sequences in comparison to each other, wherein a “homologue” is used both to refer to a homologous protein and to the gene (DNA sequence) encoding it in terms of shared ancestry. Homologous sequences are orthologous if they were separated by a speciation event: when a species diverges into two separate species, the copies of a single gene in the two resulting species are said to be orthologous. Homologous sequences are paralogous, if they were separated by a gene duplication event: if a gene in an organism is duplicated to occupy two different positions in the same genome, then the two copies are paralogous.

The term “operatively linked”, “operably linked” or “functionally linked” specifically in the context of molecular constructs, for example plasmids or expression vectors, means that one element, for example, a regulatory element, or a first protein-encoding sequence, is linked in such a way with a further part so that the protein-encoding nucleotide sequence, i.e., is positioned in such a way relative to the protein-encoding nucleotide sequence on, for example, a nucleic acid molecule that an expression of the protein-encoding nucleotide sequence under the control of the regulatory element can take place in a living cell.

The term “orthologue” as used herein refers to one of two or more homologous gene sequences found in different species.

The term “paralogue” as used herein refers to a pair of genes that derives from the same ancestral gene and now reside at different locations within the same genome.

The terms “plant”, “plant organ”, or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example, animal cells, fungal cells or plant cells, can have any degree of ploidity, i.e. they may either be haploid, diploid, tetraploid, hexaploid or polyploid.

The term “plant parts” as used herein includes, but is not limited to, isolated and/or pre-treated plant parts, including organs and cells, including protoplasts, callus, leaves, stems, roots, root tips, anthers, pistils, seeds, grains, pericarps, embryos, pollen, sporocytes, ovules, male or female gametes or gametophytes, cotyledon, hypocotyl, spike, floret, awn, lemma, shoot, tissue, petiole, cells, and meristematic cells.

A “plant material” as used herein refers to any material which can be obtained from a plant during any developmental stage. The plant material can be obtained either in planta or from an in vitro culture of the plant or a plant tissue or organ thereof. The term thus comprises plant cells, tissues and organs as well as developed plant structures as well as sub-cellular components like nucleic acids, polypeptides and all chemical plant substances or metabolites which can be found within a plant cell or compartment and/or which can be produced by the plant, or which can be obtained from an extract of any plant cell, tissue or a plant in any developmental stage. The term also comprises a derivative of the plant material, e.g., a protoplast, derived from at least one plant cell comprised by the plant material. The term therefore also comprises meristematic cells or a meristematic tissue of a plant.

As used herein “a preselected site”, “predetermined site” or “predefined site” indicates a particular nucleotide sequence in the genome (e.g. the nuclear genome, or the organellar genome, including the mitochondrial or chloroplast genome) at which location it is desired to insert, replace and/or delete one or more nucleotides. The predetermined site is thus located in a “genomic target sequence/site” of interest and can be modified in a site-directed manner using a site- or sequence-specific genome editing system.

A “Prime Editing system” as used herein refers to a system as disclosed in Anzalone et al. (2019). Search-and-replace genome editing without double-strand breaks (DSBs) or donor DNA. Nature, 1-1). Base editing as detailed above, does not cut the double-stranded DNA, but instead uses the CRISPR targeting machinery to shuttle an additional enzyme to a desired sequence, where it converts a single nucleotide into another. Many genetic traits in plants and certain susceptibility to diseases caused by plant pathogens are caused by a single nucleotide change, so base editing offers a powerful alternative for GE. But the method has intrinsic limitations and is said to introduce off-target mutations which are generally not desired for high precision GE. In contrast, Prime Editing (PE) systems steer around the shortcomings of earlier CRISPR based GE techniques by heavily modifying the Cas9 protein and the guide RNA. The altered Cas9 only “nicks” a single strand of the double helix, instead of cutting both. The new guide RNA, called a pegRNA (prime editing extended guide RNA), contains an RNA template for a new DNA sequence, to be added to the genome at the target location. That requires a second protein, attached to Cas9 or a different CRISPR effector nuclease: a reverse transcriptase enzyme, which can make a new DNA strand from the RNA template and insert it at the nicked site. To this end, an additional level of specificity is introduced into the GE system in view of the fact that a further step of target specific nucleic acid:nucleic acid hybridization is required. This may significantly reduce off-target effects. Further, the PE system may significantly increase the targeting range of a respective GE system in view of the fact that BEs cannot cover all intended nucleotide transitions/mutations (C→A, C→G, G→C, G→T, A→C, A→T, T→A, and T→G) due to the very nature of the respective systems, and the transitions as supported by BEs may require DSBs in many cell types and organisms.

As used herein, a “regulatory sequence”, or “regulatory element” refers to nucleotide sequences which are not part of the protein-encoding nucleotide sequence but mediate the expression of the protein-encoding nucleotide sequence. Regulatory elements include, for example, promoters, cis-regulatory elements, enhancers, introns or terminators. Depending on the type of regulatory element it is located on the nucleic acid molecule before (i.e., 5′ of) or after (i.e., 3′ of) the protein-encoding nucleotide sequence. Regulatory elements are functional in a living plant cell.

An “RNA-guided nuclease” is a site-specific nuclease, which requires an RNA molecule, i.e. a guide RNA, to recognize and cleave a specific target site, e.g. in genomic DNA or in RNA as target. The RNA-guided nuclease forms a nuclease complex together with the guide RNA and then recognizes and cleaves the target site in a sequence-dependent matter. RNA-guided nucleases can therefore be programmed to target a specific site by the design of the guide RNA sequence. The RNA-guided nucleases may be selected from a CRISPR/Cas system nuclease, including CRISPR/Cpf1 (CRISPR/Cas12a) systems, CRISPR/C2C2 systems, CRISPR/CasX systems, CRISPR/CasY systems, CRISPR/Cmr systems, CRISPR/Cms systems, CRISPR/MAD7 systems, CRISPR/MAD2 systems and/or any combination, variant, or catalytically active fragment thereof. Such nucleases may leave blunt or staggered ends. Further included are nickase or nuclease-dead variants of an RNA-guided nuclease, which may be used in combination with a fusion protein, or protein complex, to alter and modify the functionality of such a fusion protein, for example, in a base editor or Prime Editor.

The terms “SDN-1”, “SDN-2”, and “SDN-3” as used herein are abbreviations for the platform technique “site-directed nuclease” 1, 2, or 3, respectively, as caused by any site directed nuclease of interest, including, for example, Meganucleases, Zinc-Finger Nucleases (ZFNs), Transcription Activator Like Effector Nucleases (TALENs), and CRISPR nucleases. A “site-directed nuclease” is thus able to recognize and cut, optionally assisted by further molecules, a specific sequence in a genome or an isolate genomic sequence of interest. SDN-1 produces a double-stranded or single-stranded break in the genome of a plant without the addition of foreign DNA. For SDN-2 and SDN-3, an exogenous nucleotide template is provided to the cell during the gene editing. For SDN-2, however, no recombinant foreign DNA is inserted into the genome of a target cell, but the endogenous repair process copies, for example, a mutation as present in the template to induce a (point) mutation. In contrast, the SDN-3 mechanism uses the introduced template during repair of the DNA break so that genetic material is introduced into the genomic material. SDN-2 and SDN-3 approaches rely on the use of a donor template or repair template (RT) in trans to direct a targeted genomic modification.

A “site-specific nuclease” herein refers to a nuclease or an active fragment thereof, which is capable to specifically recognize and cleave DNA at a certain location. This location is herein also referred to as a “target sequence”. Such nucleases typically produce a double-strand break (DSB), which is then repaired by non-homologous end-joining (NHEJ) or homologous recombination (HR). Site-specific nucleases include meganucleases, homing endonucleases, zinc finger nucleases, transcription activator-like nucleases and CRISPR nucleases, or variants including nickases or nuclease-dead variants thereof.

The terms “transformation”, “transfection”, “transformed”, and “transfected” are used interchangeably herein for any kind of introduction of a material, including a nucleic acid (DNA/RNA), amino acid, chemical, metabolite, nanoparticle, microparticle and the like into at least one cell of interest by any kind of physical (e.g., bombardment), chemical or biological (e.g., Agrobacterium) way of introducing the relevant at least one material.

The term “transgenic” as used according to the present disclosure refers to a plant, plant cell, tissue, organ or material which comprises a gene or a genetic construct, comprising a “transgene” that has been transferred into the plant, the plant cell, tissue organ or material by natural means or by means of transformation techniques from another organism. The term “transgene” comprises a nucleic acid sequence, including DNA or RNA, or an amino acid sequence, or a combination or mixture thereof. Therefore, the term “transgene” is not restricted to a sequence commonly identified as “gene”, i.e. a sequence encoding a protein. It can also refer, for example, to a non-protein encoding DNA or RNA sequence, or part of a sequence. Therefore, the term “transgenic” generally implies that the respective nucleic acid or amino acid sequence is not naturally present in the respective target cell, including a plant, plant cell, tissue, organ, or material. The terms “transgene” or “transgenic” as used herein thus refer to a nucleic acid sequence or an amino acid sequence that is taken from the genome of one organism, or produced synthetically, and which is then introduced into another organism, in a transient or a stable way, by artificial techniques of molecular biology, genetics and the like.

As used herein, the term “transient” implies that the tools, including all kinds of nucleic acid (RNA and/or DNA) and polypeptide-based molecules optionally including chemical carrier molecules, are only temporarily introduced and/or expressed and afterwards degraded by the cell, whereas “stable” implies that at least one of the tools is integrated into the nuclear and/or organellar genome of the cell to be modified. “Transient expression” refers to the phenomenon where the transferred protein/polypeptide and/or nucleic acid fragment encoding the protein/polypeptide is expressed and/or active transiently in the cells and turned off and/or degraded shortly with the cell growth. Transient expression thus also implies a stably integrated construct, for example, under the control of an inducible promoter as regulatory element, to regulate expression in a fine-tuned manner by switching expression on or off.

As used herein, “upstream” indicates a location on a nucleic acid molecule which is nearer to the 5′ end of said nucleic acid molecule. Likewise, the term “downstream” refers to a location on a nucleic acid molecule which is nearer to the 3′ end of said nucleic acid molecule. For avoidance of doubt, nucleic acid molecules and their sequences are typically represented in their 5′ to 3′ direction (left to right).

The terms “vector”, or “plasmid (vector)” refer to a construct comprising, inter alia, plasmids or (plasmid) vectors, cosmids, artificial yeast- or bacterial artificial chromosomes (YACs and BACs), phagemides, bacterial phage based vectors, Agrobacterium compatible vectors, an expression cassette, isolated single-stranded or double-stranded nucleic acid sequences, comprising sequences in linear or circular form, or amino acid sequences, viral vectors, viral replicons, including modified viruses, and a combination or a mixture thereof, for introduction or transformation, transfection or transduction into any eukaryotic cell, including a plant, plant cell, tissue, organ or material according to the present disclosure. A “nucleic acid vector, for instance, is a DNA or RNA molecule, which is used to deliver foreign genetic material to a cell, where it can be transcribed and optionally translated. Preferably, the vector is a plasmid comprising multiple cloning sites. The vector may further comprise a “unique cloning site” a cloning site that occurs only once in the vector and allows insertion of DNA sequences, e.g. a nucleic acid cassette or components thereof, by use of specific restriction enzymes. A “flexible insertion site” may be a multiple cloning site, which allows insertion of the components of the nucleic acid cassette according to the invention in an arrangement, which facilitates simultaneous transcription of the components and allows activation of the RNA activation unit.

Whenever the present disclosure relates to the percentage of the homology or identity of nucleic acid or amino acid sequences, this identity implies a comparison over the entire length of the respective sequence to be compared to another, the sequence of interest or subject representing the reference sequence (e.g., in the form of a SEQ ID NO as disclosed herein) wherein these identity or homology values define those as obtained by using the EMBOSS Water Pairwise Sequence Alignments (nucleotide) programme (http://www.ebi.ac.uk/Tools/psa/emboss_water/nucleotide.html) nucleic acids or the EMBOSS Water Pairwise Sequence Alignments (protein) programme (http://www.ebi.ac.uk/Tools/psa/emboss_water/) for amino acid sequences. Those tools provided by the European Molecular Biology Laboratory (EMBL) European Bioinformatics Institute (EBI) for local sequence alignments use a modified Smith-Waterman algorithm (see http://www.ebi.ac.uk/Tools/psa/ and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

### DETAILED DESCRIPTION OF THE INVENTION

At date, various genome editing (GE) systems are available for targeted GE in model plant systems and more and more also for relevant crop plants. During the last decades, several hybrid site-specific nucleases targetable to specific genome sequences, such as Fokl, associated with zinc finger nucleases (ZFNs) or transcription activator-like effector nucleases (TALENs), and clustered regularly interspaced short palindromic repeats (CRISPR) systems opened a new era of GE in plants. Meanwhile, the RNA-guided CRISPR technology is the most universal and user-friendly tool for precisely targeted genetic manipulations applicable to almost all living species. Targeting regions of interest even in complex plant genomes is meanwhile possible for relevant crop plants. However, this is just half the way to a successful and fully targetable GE event, as the repair pathways in a plant have to be controlled to obtain a controlled and predictable GE outcome. Indeed, in plants the rate of gene knock-outs resulting from DSB repair according to the NHEJ mechanism is 30-70% in plants, reaching 100% in some cases versus the rate of knock-ins, which typically do not exceed several tenths of a percent, or several percent (see Rozov et al., Int. J. Mol. Sci., 2019, 20(13), 3371). This explains the current difficulties for plant GE and the inherent problem when implementing knock-ins, as a balance has to be found between the insertion rate and its accuracy. Means for tailoring the precise HDR response in plants in a targeted way are thus urgently needed to optimize precision GE in plant cells.

To address this objective, the present invention thus provides, in a first aspect, a method for the targeted modification of at least one genomic target sequence in at least one plant cell, wherein the method may comprise the following steps: (a) providing at least one plant cell to be modified; (b) introducing into the cell: (i) at least one plant-specific HDR booster, or a sequence encoding the same, or an orthologue, paralogue, homologue, or an active fragment thereof, or a sequence encoding the same, or a combination of at least two plant-specific HDR boosters, preferably wherein the at least one plant HDR booster comprises a consensus motif according to SEQ ID NOs: 91 to 95; (ii) at least one genome editing system comprising at least one site-specific nuclease or site-specific nickase, or a sequence encoding the same, and optionally, in the case a CRISPR system is used, at least one guide molecule, or a sequence encoding the same; and (iii) at least one repair template, or a sequence encoding the same; (c) cultivating the at least one cell under conditions allowing the expression and/or assembly of the at least one plant HDR booster, the at least one genome editing system, and the at least one repair template; and (d) obtaining at least one modified cell; and (e) optionally: obtaining at least one plant, plant tissue, organ, or seed regenerated from the at least one modified cell.

The above method thus allows using a precise genome modification system together with at least one HDR booster with plant specificity and a repair template (RT) as such necessary for precise SDN-2/-3 mediated knock-ins and modifications. By shifting the error-prone NHEJ pathway to the HDR-pathway, this allows the sequence of the repair template to be integrated at or close to a target site as identified and targeted by the genome editing system's nuclease or nickase to control the outcome of the GE event reliably by shifting the plant endogenous repair response from the NHEJ to the HDR pathway.

Notably, little is known at date on the enzymes or proteins in plant suitable as HDR boosters in the sense of the present invention. Further, plant DNA repair pathways and DNA repair pathways in mammalian and human cells significantly differ from each other so that HDR activating enzymes identified in human cells and cell lines will likely not have a counterpart with high sequence identity in the genome of relevant crop plants.

First, relevant HDR boosters and patterns to identify these in crop plant genomes thus had to be defined. As detailed in Examples 1 and 2, the inventors surprisingly found that plant proteins with HDR boosting activity can be identified via rather conserved motifs or patterns. Exemplary motifs are provided for with SEQ ID NOs: 91 to 95. This allowed the provision of various HDR boosters to be tested in accordance with the methods as disclosed herein to optimize GE in crop plants for which presently no HDR booster toolbox or systematically annotated genes being orthologues or homologous to other eukaryotic DNA repair protein encoding genes are available. Further, plant-specificity as established over long evolutionary periods is necessary for a plant-specific HDR booster in view of the specificities of plant genome repair mechanisms as inherently taking place in the nucleus or in other organelles containing genomic DNA (chloroplasts, mitochondria).

In certain embodiments, the plant-specific HDR boosters, or a combination of at least two HDR boosters or more as identified and studied herein in the context of the methods of the present invention can significantly enhance the precision of plant GE in combination with at least one RT of interest.

In certain embodiments, the methods of the present invention may further comprise an additional step following either step (d) or (e) as detailed for the above first aspect comprising: (f) screening for at least one modified plant, plant cell, plant tissue, organ, or seed carrying a desired targeted modification. Including a screening step may be helpful as intermediate step during plant breeding. In view of the fact that both the genomic target site to be modified and the RT introduced including homology arms and a certain DNA element as core part of the RT to be introduced at or near a target site of interest, the outcome of the methods of the present invention and the presence of a desired targeted modification can be determined by various PCR techniques and further techniques available in the art. In case the RT core element to be inserted is a DNA tag, or a tag encoding a protein marker or tag easily detectable or screenable, this may allow an easy screening. In a next step, the DNA test tag may then be replaced by the actual RT core element to be inserted to introduce an insertion, deletion, or exchange modification of interest.

It is an advantage of the methods disclosed herein that these can proceed completely marker-free, which may be preferably in certain breeding settings.

In one embodiment, the at least one plant-specific HDR-booster, or a sequence encoding the same, the at least one genome editing system, and at least one repair template, or a sequence encoding the same, and optional further components may be introduced into a cell in a way that the corresponding effectors are expressed in a cell as proteins/enzymes (for site-specific nucleases and variants), RNA (e.g. guide RNA), and RT (as DNA, double- and single-stranded), or the individual components may be introduced as ready effectors into a cell to be modified via transfection or bombardment so that the active complexes can interact (e.g. CRISPR nuclease and guide RNA) and thus assemble inside the cell to be modified.

In one embodiment of the methods as disclosed herein, the at least one plant-specific HDR-booster, or the orthologue, paralogue, homologue, or active fragment thereof, or the nucleic acid sequence encoding the same, is independently selected from a plant-specific COM1, ExoI, XRCC3, Radx, BRCA2, ZmChr18, or a RecQ helicase protein, or any combination thereof. Suitable plant-specific HDR boosters as identified and/or tested herein are disclosed in the attached sequence listing. More than one HDR booster may be used. In view of the fact that a very low degree of sequence conservation was observed for plant-specific HDR booster genes when performing the in silico searches as detailed in Examples 1 and 2 below, it is presently believed that it may be suitable to use an HDR booster, or a variant thereof originating from a target plant genus of interest, or being closely related thereto, or a combination thereof.

In other embodiments, and in particular in case no suitable HDR booster or orthologue or homologue phylogenetically related to the genome of the target cell can be identified for a specific plant cell to be modified, a codon-optimization and, optionally a truncation or the formation of a fusion protein with another plant-specific HDR booster can be chosen.

In one embodiment, the at least one plant-specific HDR-booster may be independently selected from the group consisting of SEQ ID NOs: 24 to 30, 33, 35, 37, 39, 41, 43, 45, 47, 49, 51, 53, 55, 57, 59, 61, 63, 65, 67, 69, 71, 73, 75, 78 to 90, or 120, or from a sequence having at least 90%, 91%, 92%, 93%, 94%, 95%, 96%, 97%, 98% or 99% sequence identity thereto, or an orthologue, paralogue, homologue, or an active fragment thereof, or a nucleic acid sequence encoding the same.

In another embodiment, the nucleic acid sequence encoding the at least one plant-specific HDR-booster may be selected from the group consisting of SEQ ID NOs: 5 to 11, 32, 34, 36, 38, 40, 42, 44, 46, 48, 50, 52, 54, 56, 58, 60, 62, 64, 66, 68, 70, 72, 74, or 119, or from a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, provided that the sequence encodes a corresponding plant-specific HDR-booster as defined above.

Preferably, a plant-specific HDR booster of the present disclosure may be codon-optimized or partially codon-optimized for the codon usage of a target plant cell to be modified, in particular, in case the at least one plant-specific HDR booster of interest does not originate from the genome of the plant cell to be modified.

In certain embodiments, at least two or more plant-specific HDR booster may be used in the methods as disclosed herein to achieve a synergistically optimized HDR pathway shift. For example, at least one COM1 booster may be combined with at least one Exo1, XRCC3, Radx, BRCA2, ZmChr18, or a RecQ helicase and the like. The more than one plant-specific HDR booster can be provided individually, or as a fusion protein. In certain embodiments, ZmChr18, a protein so far mostly only predicted for Zea mays and described for its natural activity as chromatin-remodeling protein involved in the repair of DNA damage, can also be repurposed as promising plant-specific HDR booster, alone, or in combination.

In certain embodiments of the above methods, the at least one genome editing system, the at least one repair template and/or the at least one regeneration booster, or the sequence(s) encoding the same, may be provided prior to, simultaneously with, or subsequently to providing the at least one plant-specific HDR-booster. To be functional in the methods as disclosed herein, all components or tools have to be present in their active state and, e.g. for a CRISPR system, correctly assembled. Various ways of introducing the different components are known to the skilled person and are disclosed herein. Generally, it may be preferable to reduce the transformation and/or transfection steps to a minimum to avoid undue cellular stress. The timing of introduction may depend on the component and its state (ready and active versus to be transcribed and/or translated). For example, a guide RNA due to its very nature may be less stable over time, in particular before assembling with the cognate CRISPR effector. In certain embodiments, it may thus be preferably to provide all components needed to perform the methods of the present invention in one transformation/transfection, as active molecules and/or as transcribable and/or translatable constructs, or a combination thereof. In certain embodiments, it may be preferable to provide the at least one plant-specific HDR booster before the other components to guarantee its activity before the at least one genome modification system and the at least one RT are introduced. In yet a further embodiment, when at least one regeneration booster is provided, an introduction scheme may be chosen that guarantees early activity of the at least one regeneration booster to reduce cellular stress.

In certain embodiments, at least one component needed to perform the methods of the present invention may be stably integrated as expressible construct in the genome of a plant cell to be modified. In another embodiment, all components needed to perform the methods of the present invention may be provided transiently so that these (despite the DNA tag or core element within the RT) will not integrate into the genome of a cell, which may be a preferred option regarding regulatory requirements during breeding. Any combination may be used. Preferably, the at least one HDR booster encoding sequence may be stably incorporated into the genome of a plant cell to guarantee its activity before the at least one genome modification system and the at least one RT are introduced, for instance. Usually, a regeneration booster sequence will be provided transiently to avoid a prolonged effect. In certain embodiments, a regeneration booster and any other component may be provided stably in the form of an inducible construct to be switched on and off in a targeted way.

In certain embodiments according to the methods of the present invention, the method may comprises an intermediate regeneration step before obtaining at least one modified cell. In one embodiment, the regeneration step may comprise direct meristem organogenesis, in another embodiment, the regeneration step may comprise a step of indirect callus embryogenesis or organogenesis. These intermediate regeneration steps may be particularly suitable in case a callus intermediate, or any other plant intermediate explant comprising meristematic cells, will be used during the methods of the present invention.

According to the methods as disclosed herein, the at least one plant-specific HDR booster, the at least one genome editing system, the at least one regeneration booster and/or the at least one repair template, or the sequences encoding the same, may be introduced into the cell by transformation or transfection mediated by biolistic bombardment, Agrobacterium-mediated transformation, micro- or nanoparticle delivery, chemical transfection, or any combination thereof.

Particle or biolistic bombardment may be a preferred strategy according to the methods disclosed herein, as it allows the direct and targeted introduction of exogenous nucleic acid and/or amino acid material in a precise manner not relying on the biological spread and expression of biological transformation tools, including Agrobacterium. A biological transformation technique or a chemical transformation/transfection technique as available to the skilled person may be combined with or used instead of biolistic transformation though.

In certain embodiments, the biolistic bombardment comprises a step of osmotic treatment before and/or after bombardment. Osmotic treatment can be highly suitable to enhance the transformation/transfection capacity of a cell before bombardment. Further, it can increase the transformation/transfection efficiency after bombardment. Various osmotic treatment protocols are disclosed below, and further cell-type specific protocols are available to the skilled person in the field of plant biotechnology.

The provision of at least one genome modification system, preferably a genome editing system, is necessary during the methods of the present invention to recognize and cleave a genomic target site of interest. The genome modification system may be provided together with, i.e., simultaneously, or subsequently, to one and the same target cell with the at least one HDR booster and the at least one RT, and, optionally, the at least one regeneration booster, or a regeneration booster chemical. This strategy does not only profit from the general effects of regeneration boosters on the regenerative capacity of a plant cell, the combined use may also increase genome editing efficiency in a synergistic way.

Any kind of site-directed genome editing leaves a single- or double-strand break and/or modified a certain base in a genomic target sequence of interest. This manipulation initiates stress and cellular repair responses hampering a generally high genome editing efficiency. The combined introduction of at least one genome editing system and at least one regeneration booster, or a regeneration booster chemical, can thus dramatically increase the frequency of site-directed positive (i.e., desired) genome editing events detectable throughout a high proportion of relevant target cells transformed/transfected.

In certain embodiments according to the methods disclosed herein, the methods include the introduction of at least one site-directed nuclease, nickase or even an inactivated nuclease, or a sequence encoding the same, wherein the site-directed nuclease, nickase or an inactivated nuclease may be selected from the group consisting of a CRISPR nuclease or a CRISPR system, including a CRISPR/Cas system, preferably from a CRISPR/MAD7 system, a CRISPR/Cpf1 (CRISPR/Cas12a) system, a CRISPR/MAD2 system, a CRISPR/Cas9 system, a CRISPR/CasX system, a CRISPR/CasY system, a CRISPR/Cas13 system, or a CRISPR/Csm system, a zinc finger nuclease system, a transcription activator-like nuclease system, or a meganuclease system, or any combination, variant, or an active fragment thereof. An exemplary construct is shown in SEQ ID NO: 2.

In yet a further embodiment, a CRISPR system according to the present disclosure may be used in combination with an anti-CRISPR (ACR) system (Marino et al., Nature Methods, May 2020, vol. 17, no. 5, p. 471) for providing an even better control of the activity of a CRISPR system of interest, e.g., by providing an even tighter post-translational control of the CRISPR system and/or for reducing off-target activity.

In certain embodiments, the at least one genome editing system may further comprise at least one reverse transcriptase and/or at least one cytidine or adenine deaminase, preferably wherein the at least one cytidine or adenine deaminase is independently selected from an apolipoprotein B mRNA-editing complex (APOBEC) family deaminase, preferably a rat-derived APOBEC, an activation-induced cytidine deaminase (AID), an ACF1/ASE deaminase, an ADAT family deaminase, an ADAR2 deaminase, or a PmCDA1 deaminase, a TadA derived deaminase, and/or a transposon, or a sequence encoding the aforementioned at least one enzyme, or any combination, variant, or an active fragment thereof.

A variety of suitable genome editing systems that can be employed according to the methods of the present invention, is available to the skilled person and can be easily adapted for use in the methods used herein. Various components of functional GE system, including the presence of nuclear localization signals (NLS; cf. SEQ ID NO: 2) or organellar localization signals in constructs intended to reach the nucleus or a respective organelle of interest are known and available to the skilled person.

In embodiments, wherein the site-directed nuclease or variant thereof is a nucleic acid-guided site-directed nuclease, in particular, a CRISPR nuclease, the at least one genome editing system additionally includes at least one guide molecule, or a sequence encoding the same. The “guide molecule” or “guide nucleic acid sequence” (usually called and abbreviated as guide RNA, crRNA, crRNA+tracrRNA, gRNA, sgRNA, depending on the corresponding CRISPR system representing a prototypic nucleic acid-guided site-directed nuclease system), which recognizes a target sequence to be cut by the nuclease. The at least one “guide nucleic acid sequence” or “guide molecule” comprises a “scaffold region” and a “target region”. The “scaffold region” is a sequence, to which the nucleic acid guided nuclease binds to form a targetable nuclease complex. The scaffold region may comprise direct repeats, which are recognized and processed by the nucleic acid guided nuclease to provide mature crRNA. A pegRNAs may comprise a further region within the guide molecule, the so-called “primer-binding site”. The “target region” defines the complementarity to the target site, which is intended to be cleaved. A crRNA as used herein may thus be used interchangeably herein with the term guide RNA in case it unifies the effects of meanwhile well-established CRISPR nuclease guide RNA functionalities. Certain CRISPR nucleases, e.g., Cas9, may be used by providing two individual guide nucleic acid sequences in the form of a tracrRNA and a crRNA, which may be provided separately, or linked via covalent or non-covalent bonds/interactions. The guide RNA may also be a pegRNA of a Prime Editing system as further disclosed below. The at least one guide molecule may be provided in the form of one coherent molecule, or the sequence encoding the same, or in the form of two individual molecules, e.g., crRNA and tracr RNA, or the sequences encoding the same.

In certain embodiments, the genome editing system may be a base editor (BE) system.

In yet another embodiment, the genome editing system may be a Prime Editing system.

As detailed above, the methods of the present invention rely on the provision of at least one repair template to control the genome modification event in a desired and targeted manner, as this is intended, for example, for SDN-2/SDN-3 modification and/or knock-in events. This kind of modification is particularly difficult in plant cells in view of the naturally occurring repair phenomena and the predominance of the NHEJ pathway.

Generally, according to the various aspects of the methods as disclosed herein, the at least one repair template (RT) may comprise or encode a double- (dsODN) and/or single-stranded (ssODN) nucleic acid sequence.

In certain embodiments, ssODNs may be preferred as they are more versatile and flexible. In other embodiments, dsODNs may be preferred due to their stability, and in particular for certain SDN-3 settings in case long stretch knock-ins are of interest.

Notably, the methods as disclosed herein are compatible with the use of ssODNs as well as dsODNs. Both kind of RTs can be provided as DNA synthesized ex vivo, or as expression vector, or part thereof, e.g., linearized and/or as plasmids. The choice of a ssODN and/or a dsODN may depend on the insert to be introduced and the kind of modification to be made. As detailed above, dsODNs may be preferable for long inserts due to their stability. In certain embodiments, ssODNs, likely subject to a different endogenous repair mechanism in a plant cell than dsODNS, may have the advantage of less toxicity, or a lower chance of random insertion (e.g., through NHEJ) into the genome in comparison to dsODNs.

In certain embodiments of the methods of the present invention, the at least one repair template may comprise symmetric or asymmetric homology arms, and/or the at least one repair template may comprises at least one chemically modified base and/or backbone, including a phosphothioate modified backbone, or a fluorescent marker attached to a nucleic acid of the repair template and the like. A repair template oligonucleotide may thus have at least one of a backbone modification and/or a base modification. Individual positions may be modified or added (to the 5′ and/or 3′end, or in a position in between), or the whole backbone or parts thereof may be modified.

Depending on the setting of the experiment, in which a repair template will be added to a cell of interest in the context of the present disclosure, the repair template may be provided as DNA and/or RNA, or it may be encoded on a suitable vector (e.g., a plasmid).

Any nucleic acid sequence comprised by or encoding a genetic element or construct according to the methods disclosed herein may be “codon optimized” or at least partially codon optimized for the codon usage of a plant target cell of interest. This means that the sequence is adapted to the preferred codon usage in the organism that it is to be expressed in, i.e. a “target cell of interest”, as codon optimization may increase the translation efficiency significantly.

In certain embodiments according to the methods of the present invention, the methods may further comprise during step (b) a step of (iv) providing at least one regeneration booster, or a sequence encoding the same, for promoting plant cell proliferation to assist a targeted modification of at least one genomic target sequence, optionally after expression of the regeneration booster.

To increase the genome or gene editing efficiency, the methods can not only rely on the introduction of a genome modification system, i.e., any vector or pre-assembled complex comprising nucleic acid and/or amino acid material, the methods as disclosed herein may be particularly effective in case at least one specific regeneration booster as disclosed herein is provided (introduced or, for chemicals, applied) in parallel to alleviate stress responses in a cell and to allow rapid recovery and regeneration after a manipulation.

Another problem in the targeted modification of plant genomes is that it is observed that transformed cells are less regenerable than wild type cells. These circumstances may result in poor rates of genome editing in view of the fact that the transformed/transfected material may simply not be viable enough after the introduction of the GE tools. For example, transformed cells are susceptible to programmed cell death due to presence of foreign DNA inside of the cells. Stresses arising from delivery (e.g. bombardment damage) may trigger a cell death as well. Therefore, promoting cell division is essential for the regeneration of the modified cells. Further, genome engineering efficiency is controlled largely by host cell statuses. Cells undergoing rapid cell-division, like those in plant meristem, are the most suitable recipients for genome engineering. Promoting cell division will probably increase DNA integration or modification during DNA replication and division process, and thus increase genome engineering efficiency. The particular artificially created regeneration booster polypeptides according to the present invention may have the dual function of increasing plant regeneration as well as increasing desired genome modification and gene editing outcomes.

Consequently, it may be suitable to combine genome editing with the provision of additional regeneration boosting genes/proteins to reduce the cell stress associated with the introduction of the GE tools. Suitable “regeneration boosters” as used herein may be selected based on their functions involved in promoting cell division and plant morphogenesis. In particular, a booster or booster system of interest should be compatible with a given plant without having adverse effects on plant development. The latter point is caused by the fact that naturally occurring booster proteins are usually transcription factors guiding the progression of cell differentiation at different positions in a precise manner and thus have central roles in plant development.

In one embodiment, wherein at least one regeneration booster is provided, the at least one regeneration booster may comprise at least one of an RBP encoding sequence and/or at least one PLT encoding sequence, preferably wherein the regeneration booster comprises at least one of an RBP encoding sequence, wherein the at least one regeneration booster sequence is individually selected from any one of SEQ ID NOs: 96 to 106, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or an active fragment thereof, or wherein the at least one regeneration booster sequence is encoded by a sequence individually selected from any one of SEQ ID NOs: 4, and 107 to 116, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, provided that the sequence encodes the respective regeneration booster according to SEQ ID NOs: 96 to 106 or an active fragment thereof, and optionally wherein at least one further regeneration booster is introduced, wherein the further regeneration booster, or the sequence encoding the same is selected from BBM, WUS, WOX, (Ta)RKD4, RKD2, growth regulating factors (GRF), preferably ZmGRF1/TOW according to SEQ ID NO: 118, or as encoded by SEQ ID NO: 117, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, or LEC, or any variant or active fragment of the aforementioned regeneration boosters.

Certain regeneration booster sequences, usually representing transcription factors active during various stages of plant development and also known as morphogenic regulators in plants, are known for long, including the Wuschel (WUS) and babyboom (BBM) class of boosters (Mayer, K. F. et al. Role of WUSCHEL in regulating stem cell fate in the Arabidopsis shoot meristem. Cell 95, 805-815 (1998); Yadav, R. K. et al. WUSCHEL protein movement mediates stem cell homeostasis in the Arabidopsis shoot apex. Genes Dev 25, 2025-2030 (2011); Laux, T., Mayer, K. F., Berger, J. &Jurgens, Development 122, 87-96 (1996); Leibfried, A. et al. WUSCHEL controls meristem function by direct regulation of cytokinin-inducible response regulators. Nature 438, 1172-1175 (2005); for BBM: Hofmann. The Plant Cell, Vol. 28: 1989, September 2016).

Others, including the RKD (including TaRKD4 from Triticum aestivum, or a codon-optimized version thereof) and LEC family of transcription factors have been steadily emerging and are meanwhile known to the skilled person (Hofmann, A Breakthrough in Monocot Transformation Methods The Plant Cell, Vol. 28: 1989, September 2016; New Insights into Somatic Embryogenesis: PLos one August 2013, vol. 8(8), e72160; 2015, vol. 169, pp. 2805-2821; A. Cagliari et al. New insights on the evolution of Leafy cotyledon1 (LEC1) type genes in vascular plants Genomics 103 (2014) 380-387, U.S. Pat. No. 6,825,397B1; U.S. Pat. No. 7,960,612B2, WO2016146552A1).

The Growth-Regulating Factor (GRF) family of transcription factors, which is specific to plants, is also known to the skilled person. At least nine GRF polypeptides have been identified in Arabidopsis thaliana (Kim et al. (2003) Plant J 36: 94-104), and at least twelve in Oryza sativa (Choi et al. (2004) Plant Cell Physiol 45(7): 897-904). The GRF polypeptides are characterized by the presence in their N-terminal half of at least two highly conserved domains, named after the most conserved amino acids within each domain: (i) a QLQ domain (InterPro accession IPR014978, PFAM accession PF08880), where the most conserved amino acids of the domain are Gln-Leu-Gln; and (ii) a WRC domain (InterPro accession IPR014977, PFAM accession PF08879), where the most conserved amino acids of the domain are Trp-Arg-Cys. The WRC domain further contains two distinctive structural features, namely, the WRC domain is enriched in basic amino acids Lys and Arg, and further comprises three Cys and one His residues in a conserved spacing (CX9CX10CX2H), designated as the “Effector of Transcription” (ET) domain (Ellerstrom et al. (2005) Plant Molec. Biol. 59: 663-681). The conserved spacing of cysteine and histidine residues in the ET domain is reminiscent of zinc finger (zinc-binding) proteins. In addition, a nuclear localisation signal (NLS) is usually comprised in the GRF polypeptide sequences. A preferred GRF protein suitable as regeneration booster in the methods of the present invention is GRF1 from Zea mays also known as ZmTOW (cf. SEQ ID NOs: 117 and 118). This regeneration booster is a relatively new booster recently characterized. Further suitable regeneration boosters characterized only recently and suitable for the methods as disclosed herein are disclosed in EP19183486.0.

Another class of potential regeneration boosters, yet not studied in detail for their function in artificial genome/gene editing, is the class of PLETHORS (PLT) transcription factors (Aida, M., et al. (2004). The PLETHORA genes mediate patterning of the Arabidopsis root stem cell niche. Cell 119: 109-120; Mähönen, A. P., et al. (2014). PLETHORA gradient formation mechanism separates auxin responses. Nature 515: 125-129). Organ formation in animals and plants relies on precise control of cell state transitions to turn stem cell daughters into fully differentiated cells. In plants, cells cannot rearrange due to shared cell walls. Thus, differentiation progression and the accompanying cell expansion must be tightly coordinated across tissues. PLETHORA (PLT) transcription factor gradients are unique in their ability to guide the progression of cell differentiation at different positions in the growing Arabidopsis thaliana root, which contrasts with well-described transcription factor gradients in animals specifying distinct cell fates within an essentially static context. To understand the output of the PLT gradient, we studied the gene set transcriptionally controlled by PLTs. Our work reveals how the PLT gradient can regulate cell state by region-specific induction of cell proliferation genes and repression of differentiation. Moreover, PLT targets include major patterning genes and autoregulatory feedback components, enforcing their role as master regulators of organ development (Santuari et al., 2016, DOI: https://doi.org/10.1105/tpc.16.00656). PLT, also called AIL (AINTEGUMENT-LIKE) genes, are members of the AP2 family of transcriptional regulators. Members of the AP2 family of transcription factors play important roles in cell proliferation and embryogenesis in plants (E I Ouakfaoui, S. et al., 2010, 74(4-5):313-326.). PLT genes are expressed mainly in developing tissues of shoots and roots, and are required for stem cell homeostasis, cell division and regeneration, and for patterning of organ primordia. PLT family comprises an AP2 subclade of six members. Four PLT members, PLT1/AIL3 PLT2/, AIL4, PLT3/A/L6, and BBM/PLT4/AIL2, are expressed partly overlap in root apical meristem (RAM) and required for the expression of QC (quiescent center) markers at the correct position within the stem cell niche. These genes function redundantly to maintain cell division and prevent cell differentiation in root apical meristem. Three PLT genes, PLT3/AIL6, PLT5/AIL5, and PLT7/AIL7, are expressed in shoot apical meristem (SAM), where they function redundantly in the positioning and outgrowth of lateral organs. PLT3, PLT5, and PLT7, regulate de novo shoot regeneration in Arabidopsis by controlling two distinct developmental events. PLT3, PLT5, and PLT7 required to maintain high levels of PIN1 expression at the periphery of the meristem and modulate local auxin production in the central region of the SAM which underlies phyllotactic transitions. Cumulative loss of function of these three genes causes the intermediate cell mass, callus, to be incompetent to form shoot progenitors, whereas induction of PLT5 or PLT7 can render shoot regeneration in a hormone-independent manner. PLT3, PLT5, PLT7 regulate and require the shoot-promoting factor CUP-SHAPED COTYLEDON2 (CUC2) to complete the shoot-formation program. PLT3, PLT5, and PLT7, are also expressed in lateral root founder cells, where they redundantly activate the expression of PLT1 and PLT2, and consequently regulate lateral root formation.

Regeneration boosters derived from naturally occurring transcription factors, as, for example, BBM or WUS, and variants thereof, may have the significant disadvantage that uncontrolled activity in a plant cell over a certain period of time will have deleterious effects on a plant cell. Therefore, the present invention preferably relies on the use of artificial regeneration booster proteins being the result of a series of multiple sequence alignments, domain shuffling, truncations, and codon optimization for various target plants. By focusing on core consensus motifs, new variants of regeneration boosters not occurring in nature were identified that are particularly suitable for genome modifications and gene editing. These sequences are shown in SEQ ID NOs: 4, 96 to 103, and 107 to 113. Alternatively, at least one of SEQ ID NOs: 104 to 106 and 114 to 118 may be used in certain embodiments as first or sole regeneration booster.

In yet another embodiment of the above methods, the regeneration booster may comprise at least one first RBP or PLT sequence, or a sequence encoding the same, preferably at least one RBP sequence, or the sequence encoding the same, and the regeneration booster may further comprise: (i) at least one further RBP and/or PLT sequence, or the sequence encoding the same, or a variant thereof, (ii) at least one BBM sequence, or the sequence encoding the same, or a variant thereof, (iii) at least one WOX sequence, including WUS1, WUS2, or WOX5, or the sequence encoding the same, or a variant thereof, (iv) at least one RKD4 or RKD2 sequence, including wheat RKD4, or the sequence encoding the same, or a variant thereof, (v) at least one GRF sequence, including Zea mays GRF5 and Zea mays TOW/GRF1, or the sequence encoding the same, or a variant thereof, and/or (vi) at least one LEC sequence, including LEC1 and LEC2, or the sequence encoding the same, or a variant thereof, and wherein the at least one second regeneration booster, or a sequence encoding the same, is different to the first regeneration booster. Various combinations of different regeneration booster proteins are compatible with the methods of the present invention.

In one embodiment, at least on RBP2 protein, or the sequence encoding the same, or a variant thereof may be used. In other embodiments, at least on PLT7 protein, or the sequence encoding the same, or a variant thereof may be used. In certain embodiments at least an RBP2 and a PLT7 or PLT5 protein, or the respective sequences encoding the same, or variants thereof may be used in combination. Further regeneration boosters can be added depending on the plant cell to be modified.

A “regeneration booster” as used herein may not only refer to a protein, or a sequence encoding the same, having plant proliferative activity, as defined above. A “regeneration booster” may also be a chemical added during genome modification of a plant cell of interest to be modified.

In one embodiment, the regeneration booster may thus be a chemical selected from MgCl₂ or MgSO₄, for example in a range from about 1 to 100 mM, preferably in a range from about 10 to 20 mM, spermidine in a range from about 0.1-1 mM, preferably in a range from about 0.1-0.5 mM, TSA (trichostin A), and TSA-like chemicals.

The use of at least one regeneration booster in an artificial and controlled context according to the methods disclosed herein thus has the effect of promoting plant cell proliferation. This effect is highly favourable for any kind of plant genome modification, as it promotes cell regeneration after introducing any plasmid or chemical into the at least one plant cell via transformation and/or transfection, as these interventions necessarily always cause stress to a plant cell.

In one embodiment, the various recombinant nucleic acid constructs as used according to the methods as disclosed herein may comprise at least one regulatory element as detailed below. The choice of at least one suitable regulatory element will be guided by the question of the host cell of interest and/or spatio-temporal expression patterns of interest, so that the optimum regulatory elements can be chosen to achieve a specific expression of the at least one nucleic acid sequence of interest.

In one embodiment, different promoters may be chosen, for example, the promoters having different activities so that the at least two genes can be expressed in a defined and controllable manner.

In one embodiment of the methods of the present invention, the at least one plant-specific HDR booster, the at least one genome editing system, the at least one repair template, and optionally the at least one regeneration booster, or the respective sequences encoding the same, may be introduced transiently or stably, or as a combination thereof, into a target cell to be modified.

In a further aspect, there is provided a plant, plant cell, tissue, organ, or seed which may be obtainable by, or which may be obtained by a method as defined above.

In certain embodiments, the plant may be a monocotyledonous or a dicotyledonous plant, preferably a crop plant of interest.

In certain embodiments of the aforementioned aspect, the plant may be selected from a plant originating from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant or plant cell may originate from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia foumieri, Allium cepa, Allium fistulosum, Allium sativum, Allium tuberosum, Helianthus annuus, Helianthus tuberosus and/or Spinacia oleracea.

Preferred plants, plant cells, tissues, organs, or seeds may originate from Zea spp., Beta spp., Triticum spp., Brassica spp, Solanum, Hordeum spp., and Secale spp.

In a further aspect, there is provided a generally applicable expression construct assembly, expression construct assembly, which may comprise: (i) at least one vector encoding at least one plant-specific HDR booster, preferably wherein the plant-specific HDR booster is as defined according to the first aspect above, (ii) at least one vector encoding at least one genome editing system, preferably wherein the genome editing system is as defined according to the first aspect above, optionally comprising at least one vector encoding at least one guide molecule as defined for the first aspect above guiding the at least one nucleic acid guided nuclease or nickase to the at least one genomic target site of interest; (iii) optionally: at least one vector encoding at least one repair template, preferably wherein the repair template is as defined according to the first aspect above; and (iv) optionally: at least one vector encoding at least one regeneration booster, preferably wherein the regeneration booster according to the first aspect above; wherein (i), (ii), (iii), and/or (iv) are encoded on the same, or on different vectors.

In certain embodiments of the above aspect of an expression construct assembly, the at least one RT may be provided separately as pre-synthesized ODN, or as otherwise ex vivo prepared RT. This may be useful, for example, in case the use of an ssODN may be of interest. In other embodiments, an additional at least one vector encoding the at least one RT may be provided as part of the expression construct assembly. In yet a further embodiment, a linearized plasmid may be provided, particularly, in case a dsODN as RT may be of interest.

In one embodiment, the expression construct assembly may further comprise a vector encoding at least one marker, or the marker may be additionally encoded on one of the vectors, preferably wherein the marker is introduced in a transient manner. Generally, the methods of the present invention are favourable in that regard that these methods do not need the presence of an artificial marker sequence so that the methods can be performed completely free of a marker. Still, in particular for experimental settings, the use of a marker, for example fluorescence markers or tags, may be convenient to easily study whether the methods have been conducted successfully. These markers can thus be incorporated as part of at least one RT according to the methods as disclosed herein.

A variety of suitable fluorescent marker proteins and fluorophores applicable over the whole spectrum, i.e., for all fluorescent channels of interest, for use in plant biotechnology for visualization of metabolites in different compartments are available to the skilled person, which may be used according to the present invention. Examples are GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum), an orange, a red or far-red fluorescent protein (e.g., tdTomato (tdT), or DsRed), and any of a variety of fluorescent and coloured proteins may be used depending on the target tissue or cell, or a compartment thereof, to be excited and/or visualized at a desired wavelength.

In one embodiment, the expression construct assembly comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

Notably different components of the at least one plant-specific HDR booster, a genome modification or editing system and/or a regeneration booster sequence and/or a guide molecule and/or a repair template present on the same vector of an expression vector assembly may be comprise or encode more than one regulatory sequence individually controlling transcription and/or translation.

In one embodiment of the expression construct assembly described above, the construct comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

In another embodiment of the expression construct assembly described above, the regulatory sequence comprises or encodes at least one promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

In a further embodiment of the expression construct assembly described above, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

In one embodiment of the expression construct assembly according to any of the embodiments described above, the construct comprises or encodes a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and a AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In addition, the expression construct assembly may comprise at least one terminator, which mediates transcriptional termination at the end of the expression construct or the components thereof and release of the transcript from the transcriptional complex.

In one embodiment of the expression construct assembly according to any of the embodiments described above, the regulatory sequence may comprise or encode at least one terminator selected from the group consisting of nosT, a double 35S terminator, a ZmEf1 terminator, an AtSac66 terminator, an octopine synthase (ocs) terminator, or a pAG7 terminator, or a combination thereof. A variety of further suitable promoter and/or terminator sequences for use in expression constructs for different plant cells are well known to the skilled person in the relevant field.

The methods as disclosed herein, in particular for transient particle bombardment and direct meristem regeneration, are highly effective and efficient and able to achieve single-cell origin regeneration and homogenous genome editing without a conventional selection (e.g., using an antibiotic or herbicide resistant gene).

All elements of the expression vector assembly can be individually combined and introduced into a plant cell of interest. Further, the individual elements, or all elements can be expressed in a stable or transient manner, wherein a transient introduction may be preferable. In certain embodiments, individual elements may not be provided as part of a yet to be expressed (transcribed and/or translated) expression vector, but they may be directly transfected in the active state, simultaneously or subsequently, and can form the expression vector assembly within one and the same target plant cell to be modified of interest. For example, it may be reasonable to first transform part of the expression vector assembly encoding at least one plant-specific HDR booster and/or at least one site-directed nuclease of a genome modification system, which takes some time until the construct is functionally expressed, wherein the cognate guide molecule for a CRISPR effector as part of the genome modification system may then be transfected directly in its active RNA stage and/or at least one repair template is then transfected in its active DNA stage in a separate and subsequent introduction step to be rapidly available. The at least one plant-specific HDR booster sequence and/or the at least one genome modification or editing system and/or the at least one repair template and optionally the at least one regeneration booster sequence may also be transformed as part of one vector, as part of different vectors, simultaneously, or subsequently. It may be preferable to provide the at least one repair template, or the sequence encoding the same, separately.

Generally, using of too many individual introduction steps should be avoided, and several components can be combined in one vector of the expression vector assembly, to reduce cellular stress during transformation/transfection. In certain embodiments, the individual provision of elements of the at least one plant-specific HDR booster sequence, and/or the at least one genome modification or editing system and/or the at least one guide molecule and/or the at least one repair template and/or optionally the at least one regeneration booster sequence on several vectors and in several introduction steps may be preferable in case of complex modifications relying on all elements so that all elements are functionally expressed and/or present in a cell to be active in a concerted manner. In this context, an expression construct assembly as used herein may thus only comprise at least one element to be actively transcribed and/or translated in a plant cell, whereas other elements may be provided in their active state so that the latter elements can be directly introduced into a target plant cell to be modified and will be active as soon as they have been introduced into the cell.

The present invention is further illustrated by the following non-limiting Examples.

### EXAMPLES

### Example 1: Exploring Hypothetical HDR Booster Proteins in Silico

In view of the fact that the DNA repair machinery of human an plant cells works rather different and relevant effector proteins are optimized in function to the cell they originate from, attempts were made to define new plant derived HDR booster genes and the encoded proteins to have new tools available for all kinds of genome engineering purposes in plants. Therefore, protein sequences of known HDR-associated proteins as reported, for example, in Shaked et al. supra were used as template sequences for a BLASTp search in the NCBI protein database, or in the maize genetics and genomics database (maizegdb.org). The algorithm BLASTp was used to search for non-redundant protein sequences. Using the sequences with the highest similarity among each other, they were further taken into consideration for a subsequent domain search.

Initially, a prototypic first plant-originating sequence of potential interest as plant-specific HDR booster had to be identified. To this end, sequences from different origin (mammalian or plant, for example, Arabidopsis) were used and a BLAST search against the maizegdb (maizegdb.org, tools, blast) was used to find the best hit for Zea mays as relevant crop plant. To this end, the ZmCom1 could be identified. As a next step for this (and other sequences detailed below), Ensembl Plants was used to identify suitable orthologues and paralogues in other crop species, or at different genomic locations.

It was a striking finding of the BLASTp search that the majority of sequences identified having a high predicted sequence identity stem from hypothetical plant proteins not deeper explored yet. Further, it was a major finding that there is only a very low overall sequence identity between proteins of different non-plant organisms and plants, but also between one plant genus and another. This low conservation can be explained by the high degree of specialization of the HDR repair machinery in a cell but is less favorable to easily identify orthologues/homologues by a straightforward database search.

### Example 2: Exploring Highly Conserved Motifs in HDR Boosting Proteins

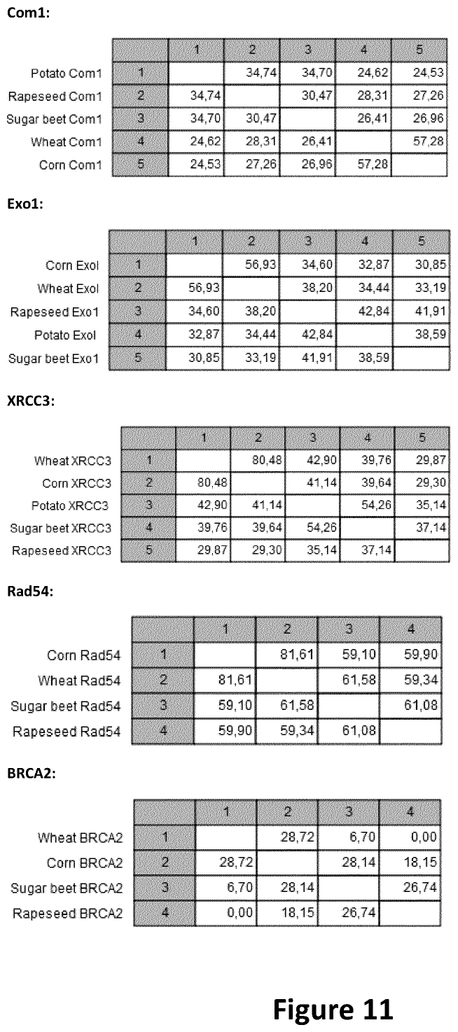

As the initial search for suitable plant HDR boosters having a desired function tuned out to be more complicated than expected, additional in silico loops were included in the hope to identify suitable proteins. To this end, orthologues should be identified using the method described in Vilella et al. (EnsemblCompara GeneTrees: complete, duplication-aware phylogenetic trees in vertebrates, Genome Res., 2009, 19: 327-335) as implemented in EnsemblePlants. It is a computer-implemented phylogenetic approach to identify protein orthologues based on the following steps:
1. Protein data set: For each species considered in the analysis, only the protein coding genes were considered. For each gene, we only consider the longest protein translation.
2. BLASTP all vs. all: Each protein was queried using WUBLASTP against each individual species protein database, including its self-species protein database.
3. Graph construction: Connections (edges) between the nodes (proteins) were retained when they satisfied either a best reciprocal hit (BRH) or a BLAST score ratio (BSR) over 0.33. A BSR for two proteins, P1 and P2, is defined as scoreP1P2/max(self-scoreP1 or self-scoreP2).
4. Clusters: We then extracted from the graph the connected components (i.e., single linkage clusters). Each connected component represents a cluster, i.e., a gene family. If the cluster has greater than 750 members, steps 3 and 4 are repeated at higher stringency (see below).
5. Multiple alignments: Proteins in the same cluster were then aligned using MUSCLE (Edgar 2004) to obtain a multiple alignment.
6. Gene tree and reconciliation: The CDS back-translated protein-based multiple alignment was used as an input to the tree program, TreeBeST, as well as the multifurcated species tree necessary for the reconciliation and the duplication calls on internal nodes.
7. Inference of orthologues and paralogs: as a next step, the resulting trees were flattened into orthologue and paralogue tables of pairwise relationships between genes. In the case of paralogues, this flattening also records the timing of the duplication due to the presence of extant species past the duplication, and thus implicitly outgroup lineages before the duplication (see FIG. 11 for results).
8. Pairwise d N/d S (nonsynonymous substitutions/synonymous substitutions): Finally, the pairwise d N/d S between pairs of genes for closely related species using codeml from the PAML package (Yang, 2007) were calculated.

The above procedure was repeated for several proteins suggested as being relevant for DNA repair in various eukaryotes and also prokaryotes, to have a sound dataset available. Based on the identified protein orthologues, next an Interproscan for protein annotation was performed to identify protein or domain classes that all respective orthologues belong to. Additionally, the different orthologues were aligned to identify specific motifs present in all identified orthologues.

As expected from early BLAST searches, the various orthologues identified only have a very low mutual sequence identity, as it shown in FIG. 11 for Com1, Exo1, XRCC3, Rad54, and BRCA2 sequences as identified and compared.

The results for the five independent searches to identify plant orthologues/homologues for HDR booster sequences can be summarized as follows:

The Interproscan revealed that ZmCom1 and all the orthologues identified (although sequence homologies were determined to be very low) belong to the protein family of DNA endonuclease RBBP8-like (IPR033316). The only overarching similarity is that all protein orthologues (plants and animals) include a SAE2 Pfam domain. This, however, was not found to be enough discriminating, as animal sequences are expected not to be functional in certain plant GE contexts.

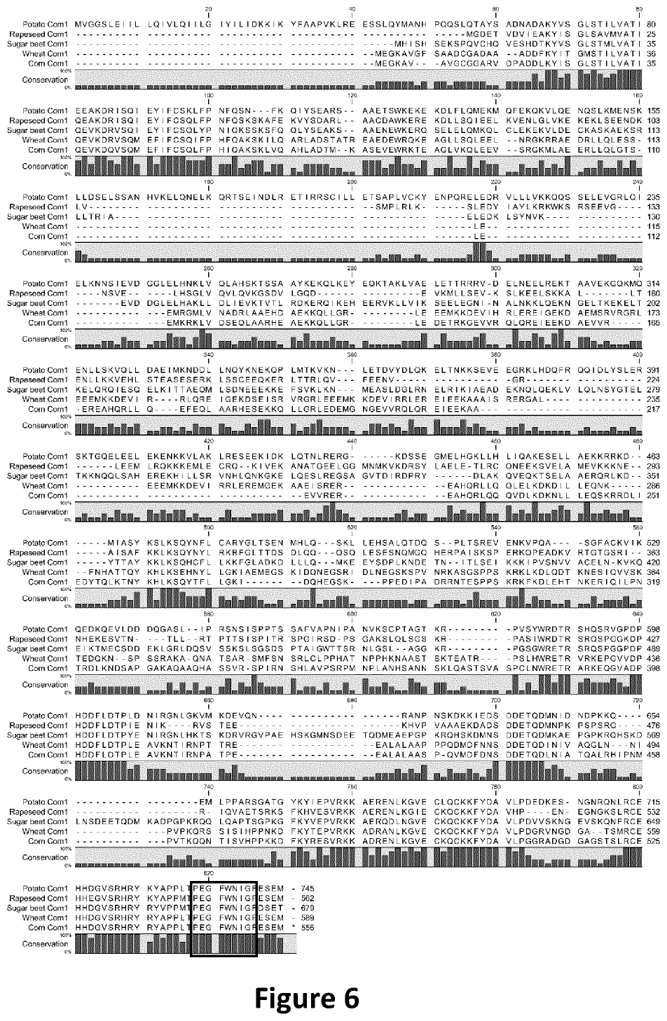

Therefore, additional searches for not yet publicly attributed motifs were made to obtain a suitable motif for plant orthologue/homologue search. Further sequences alignments and domain studies (see FIG. 6) revealed several motifs that are present in all sequences given in the sequence listing. To this end, a consensus motif/pattern was identified using the pattern identifier in CLC, which was identified to be PEGFWNIGF (SEQ ID NO: 91; FIG. 6, boxed, amino acid position 543-551 in the corn Com1 sequence). This motif cannot be identified corresponding animal orthologues, however it was identified in the corresponding soybean (SEQ ID NO: 78), rice (SEQ ID NO: 79) and grape (SEQ ID NO: 80) protein sequence and thus represents a suitable consensus sequence for the search of plant-specific COM sequences.

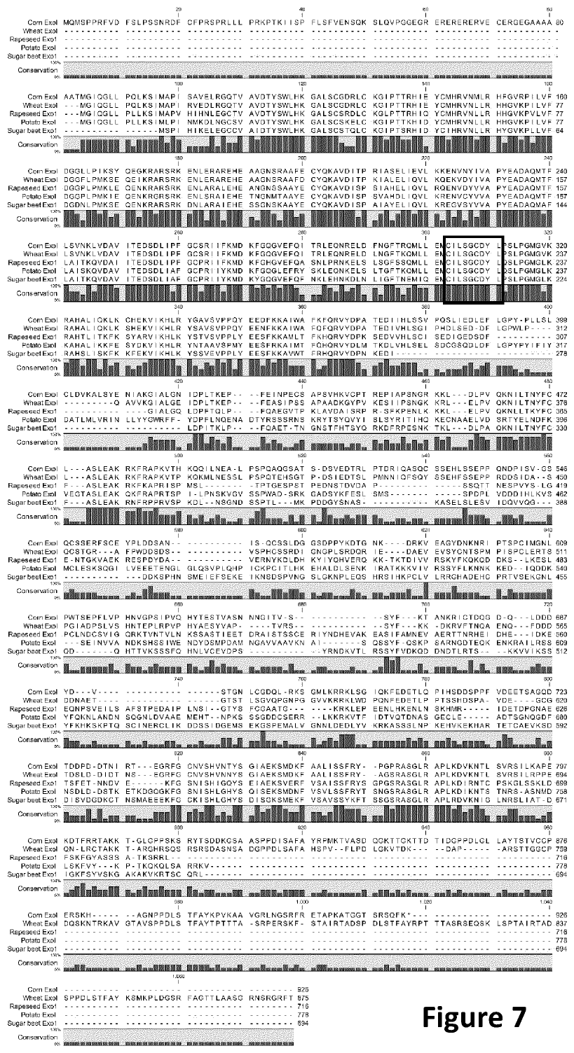

For Exo1, the Interproscan revealed that ZmExoI and all the orthologues identified (although sequence homologies were determined to be very low) belong to the protein family of Exonuclease 1 proteins (IPR032641). This overall structural classification, however, turned out not to be enough for a clear discrimination allowing the identification of plant-specific Exo1 orthologues/homologues. Sequence alignments (FIG. 7) revealed a generally highly conserved core structure. Ultimately, the pattern discovery algorithm in CLC revealed one pattern only: CILSGCDYL (SEQ ID NO: 92; FIG. 7, boxed, amino acid position 303-310 in the corn Exo1 sequence). This pattern was also identified in the corresponding rice (SEQ ID NO: 81) and Arabidopsis thaliana (SEQ ID NO: 82) protein sequence, but not in non-plant sequences of yeast etc. The corresponding Pfam domain is not clearly identified.

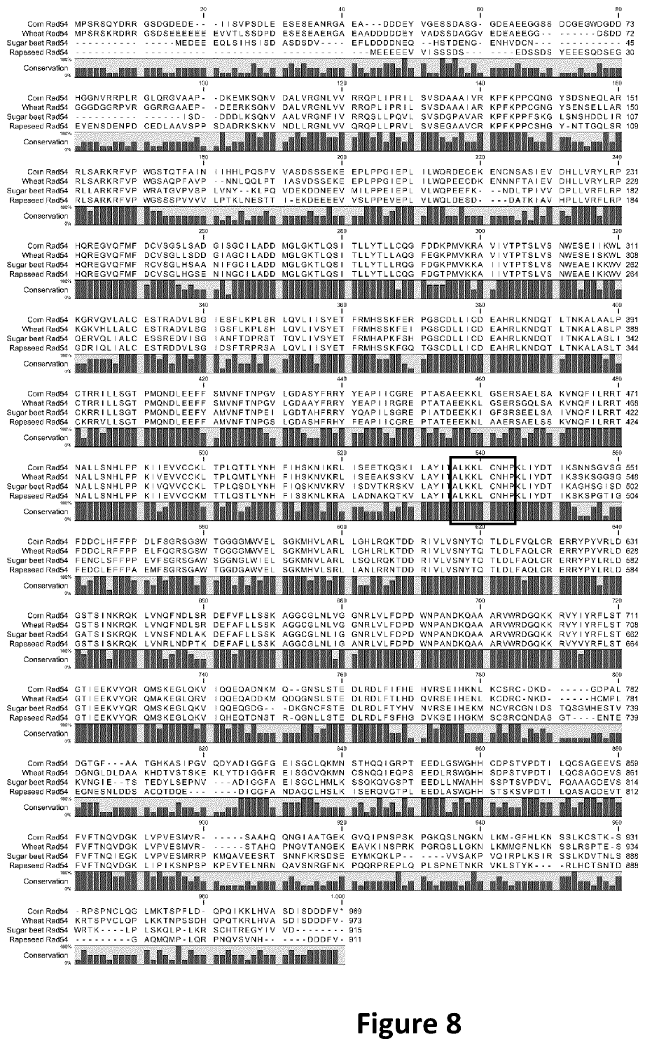

Radx searches deemed to be necessary in view of the fact that initially tested Rad52 sequences were not reliably active in certain plant systems of interest, in particular, in corn. Via an Interproscan, ZmRad54 and orthologues thereof were identified as suitable candidate, although sequence homologies were determined to be generally very low again. Rad54 as exemplary Radx belongs to the protein family of DNA REPAIR AND RECOMBINATION PROTEIN RAD54-LIKE (PTHR45629:SF6), which was as such not unexpected given the generally known function of the Rad54 proteins in their natural environment. Sequence alignments (FIG. 8) then revealed a highly conserved core structure and further zooming into and analyzing the sequences with the pattern discovery algorithm in CLC revealed one pattern only: ALKKLCNHP (SEQ ID NO: 93; FIG. 8, boxed, amino acid position 527-535 in the corn Rad54 sequence), wherein, for example, the N-terminal alanine is conserved in certain plant species, but not in the homologous Chlamydomonas sequence underpinning that the consensus motif has a discriminating nature for identifying plant sequences. This motif is part of the SNF2_N Pfam domain, but as minimum consensus sequence additionally turned out to be useful for the identification of plant Rad54 proteins suitable as HDR boosters.

For BRCA2, an Interproscan revealed that ZmBRCA2 and all the orthologues identified (again, sequence homologies between the sequences checked were determined to be very low) belong to the protein family of Breast cancer type 2 susceptibility protein (IPR015525).

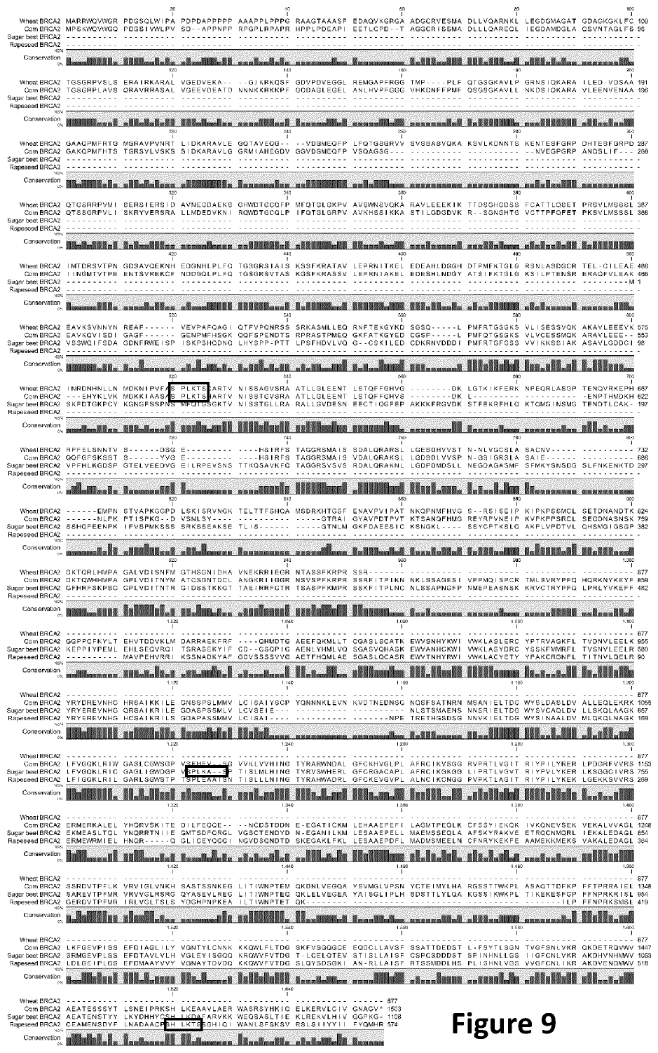

Sequence alignments (FIG. 9) revealed really low sequence identities likely not allowing an identification of suitable candidates via BLAST searches only. Therefore, a deeper pattern search was performed. We managed to identify one pattern showing plant specificity, namely: S(P/H)LK(T/A)S (SEQ ID NO: 94; FIG. 9, boxed, amino acid position 595-600 in the corn BRCA2 sequence). This motif is not part of a specific motif/Pfam domain. However, all BRCA2 orthologues given in the sequence listing have Pfam domains PF09103 and PF09169 and/or PF00634. T/A at position five of the consensus motif identified may be substituted by another aliphatic, or polar uncharged amino acid according to standard nomenclature.

The Interproscan revealed that ZmXRCC3 and all the orthologues identified (although sequence homologies were determined to be very low) possess the protein domain DNA_recomb/repair_Rad51_C (IPR013632). All except the rapeseed sequence belong to the protein family of DNA recombination and repair protein, RecA-like (IPR016467).

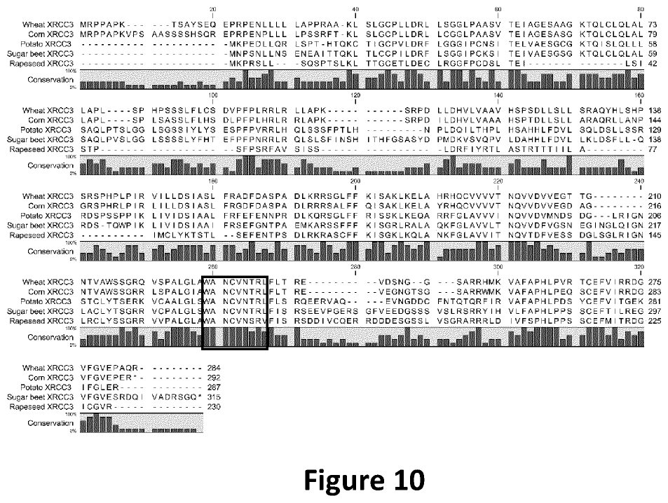

The pattern discovery algorithm in CLC revealed one pattern only: WA(N/H)CVN(S/T)R(V/L) (SEQ ID NO: 95, FIG. 10 boxed, amino acid position 229-237 in the corn XRCC3 sequence). This pattern was also identified in the corresponding Rice (SEQ ID NO: 88), Grape (SEQ ID NO: 89) and Soybean (SEQ ID NO: 90) protein sequence. This motif is part of the Pfam domain PF08423, Rad51, that is present in all orthologues of XRCC3 in the sequence listing. Still, the sub-motif consensus motif search even revealed a new consensus sequence of SEQ ID NO: 95 showing plant specificity.

For all CLC searches, CLC Main Workbench 8.0 was used relying on the parameters (Tool: Create Alignment): Gap open cost=10.0, Gap extension cost=1.0, End gap cost=Free, Alignment mode=Less accurate (fast), Redo alignments=No, Use fixpoints=No. 15% Homologies were calculated with the tool: Create Pairwise Comparison using the parameter settings: Include gaps=No, Include differences=No, Include distance=No, Include similarity=Yes, Include identity=No. For pattern discovery, the tool Pattern Discovery was used with the following parameters: Pattern length min: 4, Pattern length max: 9, Noise (%): 1.

Therefore, we succeeded in identifying new plant-specific consensus motifs allowing an identification of relevant HDR boosters. These sequences were cloned and used for further studies to evaluate the potential of the plant HDR boosters in GE SDN-2 and SDN-3 settings using a RT template for targeted repair by additionally boosting the high fidelity HDR pathway using enzymes having optimum activity in a plant cell of interest.

### Example 3: Repair Template Design

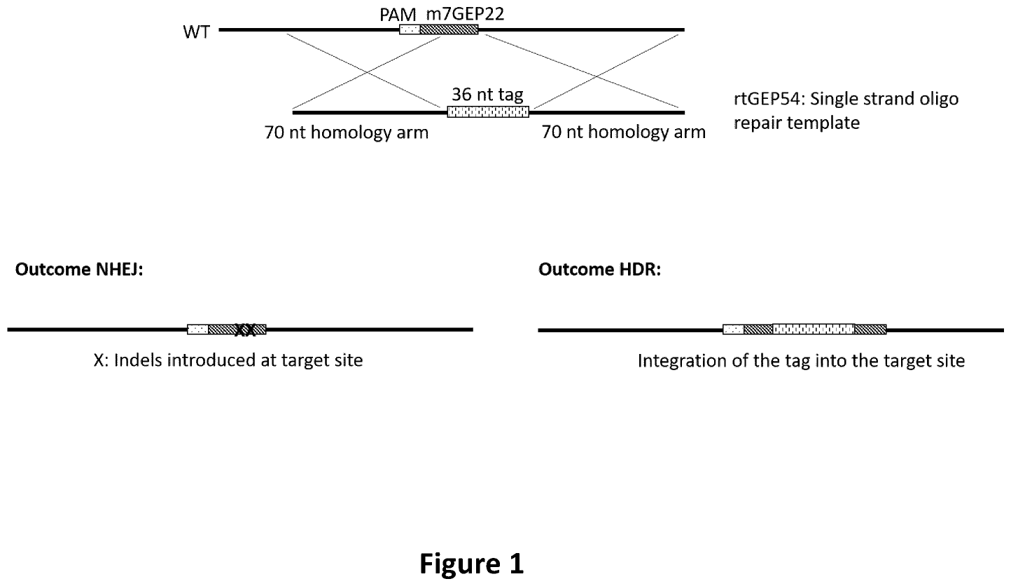

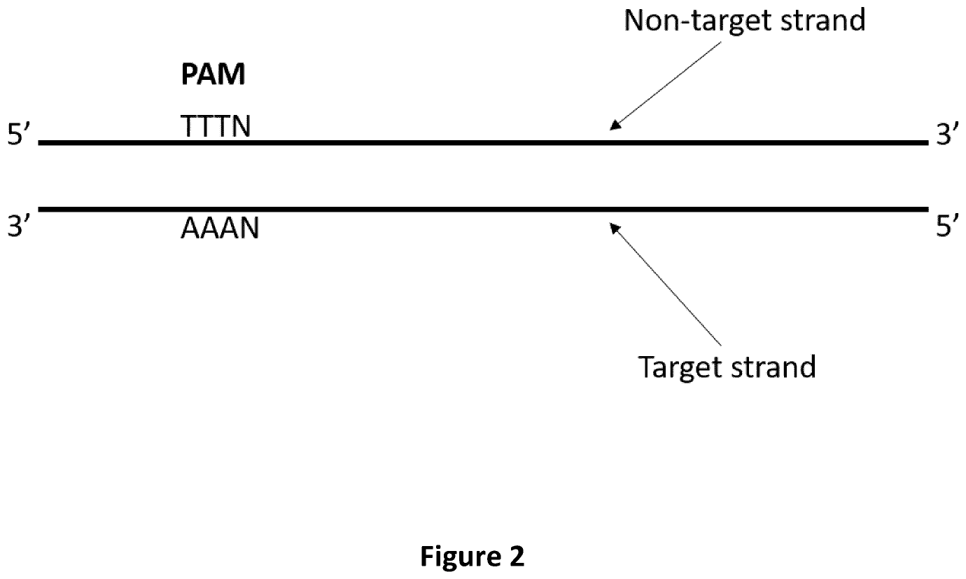

To avoid whole repair template integration, single stranded oligonucleotides of the non-target strand sequence were used as repair templates in a first series of experiments (FIG. 2). The structure of the 176 nt long repair template (rtGEP54, SEQ ID NO: 1) to target m7GEP22 in the HMG13 gene is visualized in FIG. 1. The 36 nt DNA tag sequence (SEQ ID NO: 31) to be integrated was framed with 70 nt homology arms on both sides. Additionally, PAM sites in the RT were removed by mutagenesis (TTTG to TAAG) to avoid cutting of the repair product.

For SDN-3 approaches, comparable RTs based on single stranded (ssODNs), or double stranded templates (dsODNs) can be designed as well depending on the desired outcome of the editing experiment.

To expand the scope and flexibility of our experiments, various RT variations were designed, directly synthesized (no cloning step in this high-throughput testing. RTs can obviously also be provided as (linearized) plasmid) and tested. These include RTs with generally shorter lengths and/or with asymmetric homology arms (40/70, 70/40), wherein the latter did not give good results for the moment (to be completed). Additionally, several longer RTs were designed. It could be shown in first experiments (data not shown) that 60-80 nt homology work similarly. Generally, there should be quite some flexibility in regarding the length of the homology arms. This will depend to the target site in the genome and its general accessibility, the length of the insert etc.

### Example 4: Transformation of Gene Editing Components in Maize Immature Embryo

Plasmid constructs expressing MAD7 (SEQ ID NO: 2) endonuclease, crRNA (SEQ ID NO: 3) that directs the endonuclease to the target site (e. g. m7GEP22 in HMG13), regeneration booster protein 2 (RBP2, SEQ ID NO: 4) and a single strand oligo repair template (SEQ ID NO: 1) were co-bombarded into maize immature embryos (genotype A188) using biolistic delivery. The general protocol used is as follows:

Step 1: Ear Sterilization

Maize ears with immature embryos size 0.5 to 2.5 mm were first sterilized with 10% bleach (8.25% sodium hypochlorite) plus 0.1% Tween 20 for 10 to 20 minutes, or 70% ethanol for 10-15 minutes and then washed four times with sterilized H₂O. Sterilized ears were dried briefly in a sterile hood for 5 to minutes.

Step 2: Immature Embryos Isolation for Gold Particle Bombardment

Immature embryos (preferably 1.2-1.5 mm of size, 0.8-1.8 mm also possible) were isolated under sterile conditions by first removing the top third of the kernels from the ears with a sharp scalpel. Then immature embryos were carefully pulled out of the kernel with a spatula. The freshly isolated embryos were placed onto the bombardment target area in an osmotic medium plate (N6OSM-no2,4-D medium) with scutellum-side up. Plates were sealed and incubated at 25° C. in darkness for 4-20 hours (preferably 4 hours) before bombardment.

Step 3: Bombardment

First, gold particles were prepared as follows:
1. The stock solution for gold particles can be prepared in advance, at least 1 day prior to bombardment and stored at −20° C. for at least 6 months.
2. Weigh out 10 mg of gold particles (0.4-0.6 μm) into a 1.7 ml centrifuge tube (low retention).
3. Add 1 mL of 100% ethanol (molecular biology grade), and vortex-mix for 2 min and sonicate in an ultrasonic water bath for 15 sec.
4. Pellet the gold particles by centrifuging the tube for 1 min at 3000 rpm in a bench top microcentrifuge and then discard the supernatant.
5. Repeat steps 3-4 once.
6. Add 1 ml of 70% ethanol and vortex-mix for 2 min.
7. Incubate the tube for 15 min at room temperature. Mix the contents of the tube about three times during the incubation.
8. Centrifuge the tube at 3000 rpm for 1 min, and then discard the supernatant.
9. Repeat steps 6-8 one more time.
10. Add 1 ml of sterile Milli Q water and mix for 2 min or until the particles are completely suspended.
11. Allow the particles to settle down for 1 min at room temperature and then centrifuge the tube at 3000 rpm for 1 min; discard the supernatant.
12. Repeat steps 10-11 two more times.
13. Add 1.0 ml of sterile 50% (v/v) glycerol to the gold particles at a final concentration of 10 mg/ml.
14. Store the gold particles at −20° C. until ready to use.

Notably, other established bombardment strategies and particles can be used as well depending on the system available.

Then, DNA was coated onto the gold particles (for 10 bombardments) as follows:
1. Vortex the previously prepared gold particles that were stored at −20° C. until they are completely re-suspended.
2. Pipet out 100 μl of the gold particle suspension (1 mg) into a 1.7 ml microcentrifuge tube (low retention).
3. Sonicate for 15 sec.
4. While vortex (at low to middle speed), add the following in order to each 100 μl of gold particles in 50% glycerol:
   10 μl of DNA
   100 μl of 2.5 M CaCl₂) (pre-cold on ice)
   40 μl of 0.1 M cold spermidine (prepare right before use). (Important: The order of adding gold particles, DNA, CaCl₂), and spermidine is important for the proper coating of the gold particles. Spermidine must be prepared fresh and kept on ice).
5. Close the lids and continue vortexing for 5-10 minutes at RT.
6. Allow the DNA-coated gold particles to settle 1 minute, spin for 5 seconds at the top speed, and then remove supernatant.
7. Wash the pellet in 500 μl of 100% Ethanol without disturbing pellet for 1 minute.
8. Remove supernatant without disturbing pellet. No need to spin.
9. Repeat step 7-8 one more time.
10. Finally, resuspended the DNA coated gold particles in 120 μl of 100% EtOH (for 10 bombardments). Gently vortex to resuspended the mixture.
11. Quickly pipet 10 μl of the well-suspended gold particles with a wide open 20 μl tip from the tube onto the center of a macro-carrier and spread out the gold particles around the macro-carrier evenly (note: the particles tend to form clumps at this point). Air dry and use for bombardment as soon as possible (the DNA-coated gold particles must be used within 2 h).

In a next step, the prepared gold particles were bombarded into the prepared immature embryos (osmotic treatment 4-20 h pre-bombardment by incubation on N6OSM-no2,4-D medium) using the following conditions: 3 shots per plate, 100 μg of gold particles per shot, 100 ng-500 ng of plasmid DNA per shot, 450-650 psi for 0.6 μm gold particles, at least 650 psi for 0.4 μm gold particles. After the bombardment, immature embryos were incubated for 16 to 20 h on N6OSM-no2,4-D media plates for a second osmotic treatment.

The particulars of the protocol, for example, the pressure applied will vary depending on the system used and, in particular, the plant material to be bombarded and the load to be introduced.

Step 4: Post Bombardment Culture and Regeneration

First, the formation of Type II calli was induced 16-20 h post bombardment. Therefore, embryos with dense fluorescent signals under a fluorescence microscope were selected and transferred the from N6OSM-no2,4-D onto a N6-5Ag plate (˜15 embryos per plate) with scutellum-face-up. The embryos on the N6-5Ag plate were incubated at 27° C. in darkness for 14-16 days to induce type II calli. For type II callus regeneration calli from the bombarded region of the plate were transferred to MRM1 medium and cultured on MRM1 medium at 25° C. in darkness until the somatic embryo matured (˜2 weeks). The mature somatic embryos were then transferred onto MSO medium in a phytotray for embryo germination. Therefore, they were cultured in the full light chamber at 25° C. until the plants are ready for moving to the greenhouse (˜1 week).

Media used:

N6-5Ag: N6 salt+N6 vitamin+1.0 mg/L of 2, 4-D+100 mg/L of Caseine+2.9 g/L of L-proline+20 g/L sucrose+5 g/L of glucose+5 mg/L of AgNO3+8 g/L of Bacto-agar, pH 5.8.

N6OSM-no2,4-D medium: N6+100 mg/L of Caseine+0.7 g/L of L-proline+0.2 M Mannitol (36.4 g/L)+0.2 M sorbitol (36.4 g/L)+20 g/L sucrose+15 g/L of Bacto-agar, pH 5.8.

MRM1: MS Salts+MS vitamins+100 mg/L of myoinositol+6% sucrose+9 g/L of Bactoagar, pH 5.8.

MSO: MS Salts+MS vitamins+2 g/L of myoinositol+2% sucrose+8 g/L of Bactoagar, pH 5.

### Example 5: Detection of HDR-Mediated Gene Editing at the m7GEP22 Target Site

Two weeks after bombardment, at the end of the type-II callus induction phase, type-II calluses from each bombardment plate were collected. Calluses developed from immature embryos on the same plate were pooled as one sample. Genomic DNA was extracted from each sample and ddPCR analysis was performed to detect the percentage of all mutations, including HDR- and NHEJ-mediated events at the target site. NHEJ events were determined using a drop-off assay. The percentage of HDR-mediated events was determined with a direct quantification assay. The ratio of HDR-mediated events over all mutations was used as normalized HDR-mediated gene editing efficiency.

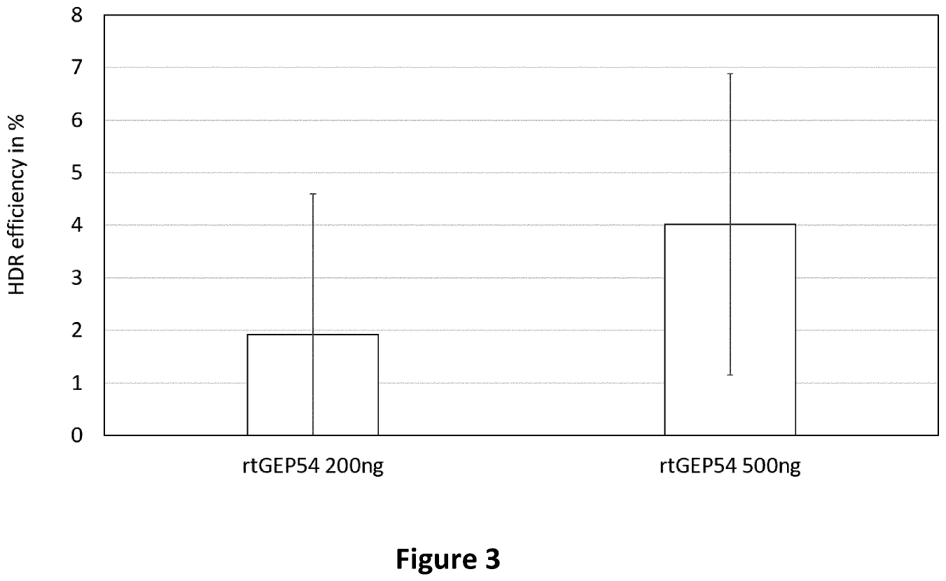

Using 200 ng of the RT for bombardment, an average HDR efficiency (HDR events/all mutations) of 1.91% was observed (FIG. 3 left column). Using 500 ng for each bombardment, HDR efficiency (HDR events/all mutations) increased to 4.01% (FIG. 3 right).

### Example 6: Boosting HDR Gene Editing Efficiencies by Overexpression of Different HDR Repair Pathway Boosting Proteins

In order to further increase the efficiency of precise HDR-mediated gene editing, multiple gene candidates that might be positively involved in the HDR pathway were searched for (see Examples 1 and 2 above), cloned and tested subsequently. These included ZmRad52 (SEQ ID NO: 5; N-terminus deleted to remove the predicted mitochondria localization signal), ZmBRCA2 (SEQ ID NO: 6), ZmCOM1 (SEQ ID NO: 7), ZmRad54 (SEQ ID NO: 8), ZmXRCC3 (SEQ ID NO: 9), ZmExoI (SEQ ID NO: 10), ZmChr18 (SEQ ID NO: 119), and RecQl4 (SEQ ID NO: 11; codon optimized for Zea mays) as RecQ helicase. Respective coding sequences were synthesized and cloned in the expression vector GEMT130 (SEQ ID NO: 12), under a ZmUbi promoter (SEQ ID NO: 13).

The generated expression vectors (SEQ ID NOs: 17 to 23) encoding for the different HDR boosting proteins were co-bombarded with plasmid constructs expressing MAD7 (SEQ ID NO: 2, plasmid: SEQ ID NO: 14) endonuclease, the crRNA (SEQ ID NO: 3, plasmid SEQ ID NO: 15) that directs the endonuclease to the target site m7GEP22, regeneration booster protein 2 (RBP2, SEQ ID NO: 4, plasmid SEQ ID NO: 16) and the single strand oligo repair template (ssODN RT) (SEQ ID NO: 1, 200 ng or 500 ng) into maize immature embryos using biolistic delivery (see Example 4). All components were precipitated on the same gold particles. HDR efficiencies were determined according to the procedure described in Example 5.

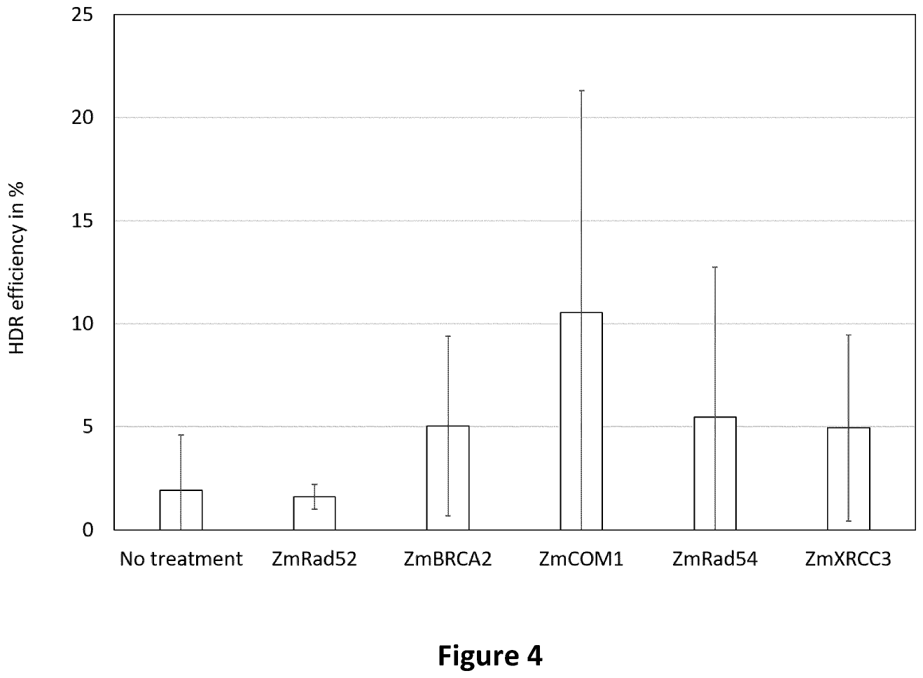

Co-bombarded cells that transiently overexpressed ZmBRCA2 (SEQ ID NO: 6), ZmCOM1 (SEQ ID NO: 7), ZmRad54 (SEQ ID NO: 8) and ZmXRCC3 (SEQ ID NO: 9) using 200 ng RT showed an increase of HDR gene editing efficiencies (HDR events/all mutations) ranging from 2 to 5 folds (FIG. 4).

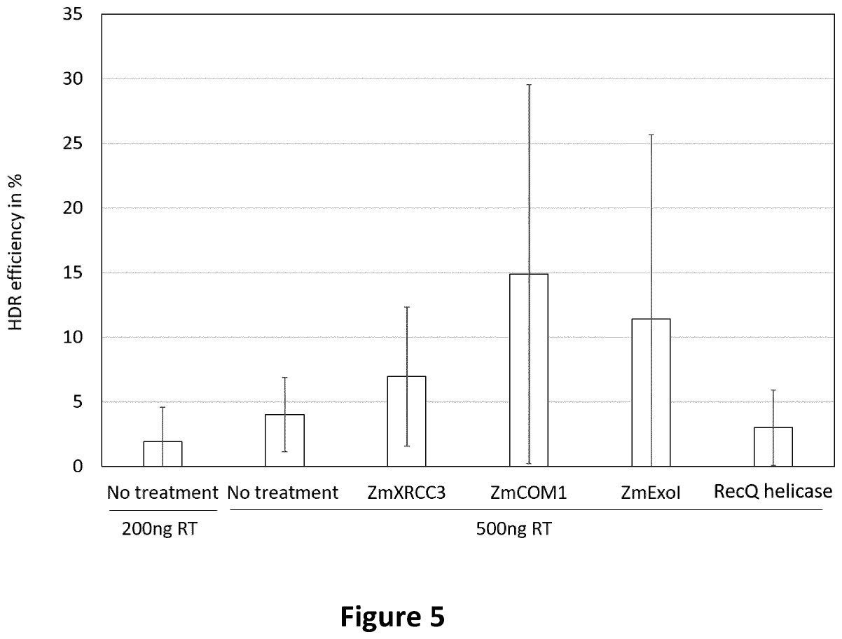

Transient overexpression of ZmXRCC3 (SEQ ID NO: 9), ZmCOM1 (SEQ ID NO: 7), ZmExoI (SEQ ID NO: 10), or RecQl4 (SEQ ID NO: 11) together with using 500 ng ssODN RT further increased HDR gene editing efficiencies (HDR events/all mutations; see FIG. 5) ranging from around 7 to around 15 folds. Transient overexpression of ZmCOM1 gave HDR efficiencies of 14.88% (SEQ ID NO: 7). ZmXRCC3 (SEQ ID NO: 9) and ZmExoI (SEQ ID NO: 10) gave HDR efficiencies of 6.96 and 11.43%, respectively. Therefore, there seems to be a dosage effect directly correlated with the amount of RT used together with a plant specific HDR booster during editing.

### Example 7: Generation of HDR-Repaired Plants with Targeted HiBiT-Tag Integration

The construct overexpressing ZmCom1 (SEQ ID NO: 19) was co-bombarded with plasmid constructs expressing MAD7 (SEQ ID NO: 2, plasmid: SEQ ID NO: 14) endonuclease, the crRNA (SEQ ID NO: 76) that directs the endonuclease to the target site crGEP43, regeneration booster protein 2 (RBP2, SEQ ID NO: 4, plasmid SEQ ID NO: 16) and the single strand oligo repair template (SEQ ID NO: 77, 500 ng) into maize immature embryos using biolistic delivery (see protocol above). All components were precipitated on the same gold particles.

Leaves of the single regenerated plants were collected for DNA extraction. Editing at the target site was analyzed using ddPCR as described above and/or using Sanger sequencing.

Overall SDN-1/2 efficiencies were determined to be between 0.7-0.9%. This corresponds to a two- to threefold increase to previously published results (Svitashev et al., 2015, Plant Physiology, vol. 169, 931-945) where similar settings—but lacking an HDR booster—were used and efficiencies of 0.3 to 0.4% were calculated. This underpins the effect of the plant-specific HDR boosters as characterized and used herein.

### Example 8: Boosting HDR Gene Editing Efficiencies by Combining Different HDR Repair Pathway Boosting Proteins

With early tests, it was observed that different combinations of HDR boosting proteins can be overexpressed to achieve improved HDR efficiencies. Therefore, further assays were planned where the repair template, the crRNA, the nuclease and different combinations (ZmCOM1+ExoI, ZmXRCC3+ZmCOM1, ZmXRCC3+ExoI and ZmCOM1+ExoI+XRCC3) of HDR booster proteins are co-bombarded. HDR gene editing efficiencies (HDR events/all mutations) should then be determined as already described in the above Examples.

Particularly encouraged by the results observed in the experiment detailed in Example 6 above, namely those results showing that transient overexpression of ZmCOM1 (SEQ ID NO: 7) and ZmExoI (SEQ ID NO: 10) had the best effects on boosting HDR efficiency, the effect of a combination of ZmCOM1 and ZmExoI was evaluated at the target site m7GEP22 to evaluate, whether the purposive combination of boosters would lead to a further and unexpected increase on boosting HDR efficiency in a positive way.

The construct overexpressing ZmCOM1 (SEQ ID NO: 19) and the construct overexpressing ZmExoI (SEQ ID NO: 23) were co-bombarded with plasmid constructs expressing MAD7 endonuclease (SEQ ID NO: 2, plasmid: SEQ ID NO: 14), the crRNA (SEQ ID NO: 3) that directs the endonuclease to the target site m7GEP22, regeneration booster protein 2 (RBP2, SEQ ID NO: 4, plasmid SEQ ID NO: 16) and a single strand oligo repair template (rtGEP67, SEQ ID NO: 121, 500 ng, 150 bp homology arms) into maize immature embryos using biolistic delivery (see protocol above in Example 4).

All components were precipitated on the same gold particles.

Leaves of the single regenerated plants were collected for DNA extraction. Editing at the target site was analyzed using qPCR with a drop-off assay (as described above for the ddPCR method, see Example 5) and/or using Sanger sequencing.

Overall HDR efficiencies were determined to be 13.1%±8.6% (n=8, meaning 8 experiments in total were performed), with a maximum observed efficiency of 25% (HDR events/all mutations). This translates into an efficiency of HDR events/total initial immature embryos of 0.3%-1.67% in the experiments with positive HDR events, meaning an improvement of up to 5,6 times when compared to HDR efficiencies without using any HDR boosters (see Svitashev et al., supra, confirming that 0.3% to 0.4% HDR efficiency can be seen as good control in experiments without boosters).

HDR-positive single plants were maintained in the greenhouse under normal growth conditions and selfed for T1 seed productions. T1 seedlings analyzed using Sanger sequencing further confirmed that these plants were either mono-allelic or bi-allelic with the expected HDR editing outcome. No chimerism was observed.

In sum, there was thus a significant increase in HDR efficiencies for the combined booster approach.

Motivated by this finding, further combinations of HDR boosters can be combined with specific combinations of regeneration boosters. For example, a single HDR booster, or a specific combination of HDR boosters can be combined with a specific combination of regeneration boosters. Preferably, at least one RBP, e.g. RBP2, and/or a PLT (e.g. PLT7) is used in combination with a single HDR booster or a combination thereof and an increase in transformation and editing efficiency is determined as detailed above.

Presently, various different targets sites are being tested to evaluate the general applicability over the area of entire plant genomes.

### Example 9: Boosting HDR Gene Editing Efficiencies by Covalent/Non-Covalent Linkage of the HDR Boosting Protein to the Nuclease

The HDR boosting proteins can be linked to the nuclease to achieve improved HDR efficiencies. Therefore, the repair template, the crRNA, a nuclease and the different fusion constructs (nuclease+HDR booster) are co-bombarded. HDR gene editing efficiencies (HDR events/all mutations) are determined as described above.

By linking the HDR booster to the nuclease, both effector enzymes are brought into close proximity exactly at the predetermined site in the genome where the site-directed nuclease will introduce a cut or nick to be repaired precisely, as assisted by the HDR booster.

1. A method for the targeted modification of at least one genomic target sequence in at least one plant cell, wherein the method comprises the following steps:
(a) providing at least one plant cell to be modified;
(b) introducing into the cell:
(i) at least one plant-specific HDR booster, or a sequence encoding the same, or an orthologue, paralogue, homologue, or an active fragment thereof, or a sequence encoding the same, or a combination of at least two plant-specific HDR boosters, preferably wherein the at least one plant HDR booster comprises a consensus motif according to SEQ ID NOs: 91 to 95;
(ii) at least one genome editing system comprising at least one site-specific nuclease or site-specific nickase, or a sequence encoding the same, and optionally, in the case a CRISPR system is used, at least one guide molecule, or a sequence encoding the same; and
(iii) at least one repair template, or a sequence encoding the same;
(c) cultivating the at least one cell under conditions allowing the expression and/or assembly of the at least one plant HDR booster, the at least one genome editing system, and the at least one repair template; and
(d) obtaining at least one modified cell; and
(e) optionally: obtaining at least one plant, plant tissue, organ, or seed regenerated from the at least one modified cell.

2. The method according to claim 1, further comprising an additional step following either step (d) or (e) comprising:
(f) screening for at least one modified plant, plant cell, plant tissue, organ, or seed carrying a desired targeted modification.

3. The method according to claim 1, the method further comprising during step (b)
(iv) providing at least one regeneration booster, or a sequence encoding the same, for promoting plant cell proliferation to assist a targeted modification of at least one genomic target sequence, optionally after expression of the regeneration booster.

4. The method according to claim 1, wherein the at least one plant-specific HDR booster, or the orthologue, paralogue, homologue, or active fragment thereof, or the nucleic acid sequence encoding the same, is independently selected from a plant-specific COM1, ExoI, XRCC3, Radx, BRCA2, ZmChr18, or a RecQ helicase protein, or any combination thereof.

5. The method according to claim 1, wherein the at least one plant-specific HDR booster is independently selected from the group consisting of SEQ ID NOs: 24 to 30, 33, 35, 37, 39, 41, 43, 45, 47, 49, 51, 53, 55, 57, 59, 61, 63, 65, 67, 69, 71, 73, 75, 78 to 90, or 120, or a sequence having at least 90%, 91%, 92%, 93%, 94%, 95%, 96%, 97%, 98% or 99% sequence identity thereto, or an orthologue, paralogue, homologue, or an active fragment thereof, or a nucleic acid sequence encoding the same.

6. The method according to claim 5, wherein the nucleic acid sequence encoding the at least one plant-specific HDR booster is selected from the group consisting of SEQ ID NOs: 5 to 11, 32, 34, 36, 38, 40, 42, 44, 46, 48, 50, 52, 54, 56, 58, 60, 62, 64, 66, 68, 70, 72, 74, or 119, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, provided that the sequence encodes a corresponding plant-specific HDR booster as defined in claim 5.

7. The method according to claim 1, wherein the at least one genome editing system, the at least one repair template and/or the at least one regeneration booster, or the sequence(s) encoding the same, is/are provided prior to, simultaneously with, or subsequently to providing the at least one plant-specific HDR booster.

8. The method according to claim 1, wherein the method comprises an intermediate regeneration step before obtaining at least one modified cell, and wherein the regeneration step comprises direct meristem organogenesis, or wherein the regeneration step comprises a step of indirect callus embryogenesis or organogenesis.

9. The method according to claim 1, wherein the at least one plant-specific HDR booster, the at least one genome editing system, the at least one regeneration booster and/or the at least one repair template, or the sequences encoding the same, are introduced into the cell by transformation or transfection mediated by biolistic bombardment, Agrobacterium-mediated transformation, micro- or nanoparticle delivery, chemical transfection, or a combination thereof, preferably wherein the introduction is mediated by biolistic bombardment, preferably wherein the biolistic bombardment comprises a step of osmotic treatment before and/or after bombardment.

10. The method according to claim 1, wherein the at least one genome editing system is selected from a CRISPR/Cas system, preferably from a CRISPR/MAD7 system, a CRISPR/Cpf1 (CRISPR/Cas12a) system, a CRISPR/MAD2 system, a CRISPR/Cas9 system, a CRISPR/CasX system, a CRISPR/CasY system, a CRISPR/Cas13 system, or a CRISPR/Csm system, or wherein the at least one site-directed nuclease or nickase, or a sequence encoding the same, is selected from a zinc finger nuclease system, or a transcription activator-like nuclease system, or a meganuclease system, or any combination, variant, or an active fragment thereof.

11. The method according to claim 1 or 10, wherein the at least one genome editing system further comprises at least one reverse transcriptase and/or at least one cytidine or adenine deaminase, preferably wherein the at least one cytidine or adenine deaminase is independently selected from an apolipoprotein B mRNA-editing complex (APOBEC) family deaminase, preferably a rat-derived APOBEC, an activation-induced cytidine deaminase (AID), an ACF1/ASE deaminase, an ADAT family deaminase, an ADAR2 deaminase, a PmCDA1 deaminase, a TadA derived deaminase, and/or a transposon, or a sequence encoding the aforementioned at least one enzyme, or any combination, variant, or an active fragment thereof.

12. The method according to claim 1, wherein the at least one repair template comprises or encodes a double- and/or single-stranded nucleic acid sequence.

13. The method according to claim 12, wherein the at least one repair template comprises symmetric or asymmetric homology arms, and/or wherein the at least one repair template comprises at least one chemically modified base and/or backbone.

14. The method according to claim 1, wherein at least one regeneration booster is provided and wherein the regeneration booster comprises at least one of an RBP encoding sequence and/or at least one PLT encoding sequence, preferably wherein the regeneration booster comprises at least one of an RBP encoding sequence, wherein the at least one regeneration booster sequence is individually selected from any one of SEQ ID NOs: 96 to 106 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or an active fragment thereof, or wherein the at least one regeneration booster sequence is encoded by a sequence individually selected from any one of SEQ ID NOs: 4, 107 to 116 or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, provided that the sequence encodes the respective regeneration booster according to SEQ ID NOs: 96 to 106 or an active fragment thereof, and optionally wherein at least one further regeneration booster is introduced, wherein the further regeneration booster, or the sequence encoding the same is selected from BBM, WUS, WOX, RKD4, RKD2, GRF, LEC, or a variant or active fragment thereof.

15. The method according to claim 14, wherein the regeneration booster comprises at least one first RBP or PLT sequence, or a sequence encoding the same, preferably at least one RBP sequence, or the sequence encoding the same, and wherein the regeneration booster further comprises:
(i) at least one further RBP and/or PLT sequence, or the sequence encoding the same, or a variant thereof,
(ii) at least one BBM sequence, or the sequence encoding the same, or a variant thereof,
(iii) at least one WOX sequence, including WUS1, WUS2, or WOX5, or the sequence encoding the same, or a variant thereof,
(iv) at least one RKD4 or RKD2 sequence, including wheat RKD4, or the sequence encoding the same, or a variant thereof,
(v) at least one GRF sequence, including Zea mays GRF5 and Zea mays GRF1/TOW, or the sequence encoding the same, or a variant thereof, and/or
(vi) at least one LEC sequence, including LEC1 and LEC2, or the sequence encoding the same, or a variant thereof,
and wherein the at least one second regeneration booster, or a sequence encoding the same, is different to the first regeneration booster.

16. The method of claim 1, wherein the at least one plant-specific HDR booster, the at least one genome editing system, the at least one repair template, and optionally the at least one regeneration booster, or the respective sequences encoding the same, are introduced transiently or stably, or as a combination thereof.

17. A plant, plant cell, tissue, organ, or seed obtainable by or obtained by a method according to claim 1.

18. The plant, plant cell, tissue, organ, or seed according to claim 17, wherein the plant is a monocotyledonous or a dicotyledonous plant.

19. The plant, plant cell, tissue, organ, or seed according to claim 17, wherein the plant is selected from a plant originating from a genus selected from the group consisting of Hordeum, Sorghum, Saccharum, Zea, Setaria, Oryza, Triticum, Secale, Triticale, Malta, Brachypodium, Aegilops, Daucus, Beta, Eucalyptus, Nicotiana, Solanum, Coffea, Vitis, Erythrante, Genlisea, Cucumis, Marta, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant or plant cell originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malta domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus and/or Spinacia oleracea.

20. An expression construct assembly, comprising:
(i) at least one vector encoding at least one plant-specific HDR booster, preferably wherein the plant-specific HDR booster is as defined in claim 1,
(ii) at least one vector encoding at least one genome editing system, preferably wherein the genome editing system is as defined in claim 1, optionally comprising at least one vector encoding at least one guide molecule as defined in claim 1 guiding the at least one nucleic acid guided nuclease or nickase to the at least one genomic target site of interest;
(iii) optionally: at least one vector encoding at least one repair template, preferably wherein the repair template is as defined in claim 1; and
(iv) optionally: at least one vector encoding at least one regeneration booster, preferably wherein the regeneration booster is as defined in claim 1;
wherein (i), (ii), (iii), and/or (iv) are encoded on the same, or on different vectors.

21. The expression construct assembly according to claim 20, wherein at least one vector of the assembly further comprises a nucleic acid sequence encoding at least one marker.
